(19) United States
(12) Patent Application Publication
(10) Pub. No.: US 2025/0214110 A1
(43) Pub. Date: Jul. 3, 2025

(54) ULTRASONIC WAVE UNIT, DIFFRACTION SWELLING TAPE, AND ULTRASONIC WAVE FOCUSING DEVICE

(71) Applicant: TOHOKU UNIVERSITY, Sendai-shi, Miyagi (JP)

(72) Inventors: Noriko TSURUOKA, Sendai-shi (JP); Yoichi HAGA, Sendai-shi (JP); Takashi SEKI, Sendai-shi (JP)

(21) Appl. No.: 18/850,977

(22) PCT Filed: Mar. 30, 2023
(86) PCT No.: PCT/JP2023/013107

§ 371 Date: Sep. 25, 2024

(30) Foreign Application Priority Data
Mar. 30, 2022 (JP) 2022-055687

(51) Int. Cl. B06B 1/06; B06B 1/02; B06B 3/04; G10K 11/28

(52) U.S. Cl. CPC B06B 1/0622; B06B 1/0238; B06B 3/04; G10K 11/28

(57) ABSTRACT

An ultrasonic wave unit includes a piezoelectric element having an ultrasonic wave generation surface for generating an ultrasonic wave, and a transmissive diffraction portion positioned on the ultrasonic wave generation surface or away from the ultrasonic wave generation surface.

### CROSS-REFERENCE TO RELATED APPLICATIONS

This is the U.S. National Phase Application under 35 U.S. C. International Patent Application No. PCT/JP2023/013107, filed Mar. 30, 2023, which claims the benefit of Japanese Patent Application No. 2022-055687, filed Mar. 30, 2022, both of which are incorporated by reference herein in their entireties for all purposes.

### TECHNICAL FIELD

the invention relates to an ultrasonic wave unit, a diffraction swelling tape, and an ultrasonic wave focusing device.

### BACKGROUND ART

For example, as disclosed in Patent Literature 1, a focusing-type sound wave therapy device including a planar-shape element for generating ultrasonic waves and an acoustic lens for transmitting the ultrasonic waves is known. In such a focusing-type sound wave therapy device, sound waves are generated in a state in which a sound wave transmission gel and the acoustic lens are interposed between the planar-shape element and a skin surface. In this way, energy of the sound waves can be focused on a treatment site at a desired depth from the skin surface.

The acoustic lens has a concave surface positioned on a side opposite to a surface attached to the planar-shape element. The concave surface has a spherical surface designed based on a curvature corresponding to a focal length from the skin to the treatment site. The focal length can be adjusted by adjusting the curvature of the concave surface.

### CITATION LIST

### Patent Literature

Patent Literature 1: JP3216192U

### SUMMARY OF INVENTION

### Technical Problem

However, in the configuration of the above-described focusing-type sound wave therapy device, it is necessary to form the concave surface on the acoustic lens. It is very difficult to process a spherical surface having a highly accurate curvature into a concave surface, and it is difficult to reduce a processing cost. Therefore, the acoustic lens having a concave surface has a problem in that it is not suitable for mass production.

The invention has been made in view of the above problem, and an object of the invention is to provide an ultrasonic wave unit, a diffraction swelling tape, and an ultrasonic wave focusing device that can reduce a processing cost by having a structure that can be easily processed.

### Solution to Problem

An ultrasonic wave unit according to one aspect of the invention includes a piezoelectric element having an ultrasonic wave generation surface for generating an ultrasonic wave, and a transmissive diffraction portion positioned on the ultrasonic wave generation surface or away from the ultrasonic wave generation surface.

In the ultrasonic wave unit according to one aspect of the invention, the transmissive diffraction portion may be positioned on the ultrasonic wave generation surface, and the transmissive diffraction portion may be a member different from the piezoelectric element.

The ultrasonic wave unit according to one aspect of the invention may include a swelling body provided on the ultrasonic wave generation surface to cover the transmissive diffraction portion.

In the ultrasonic wave unit according to one aspect of the invention, the transmissive diffraction portion may be positioned away from the ultrasonic wave generation surface, the transmissive diffraction portion may have a first surface facing and away from the ultrasonic wave generation surface and a second surface opposite to the first surface, a swelling body may be disposed at least between the ultrasonic wave generation surface and the first surface, and the swelling body may be configured to adhere at least the transmissive diffraction portion to the piezoelectric element.

In the ultrasonic wave unit according to one aspect of the invention, the swelling body may be configured to adhere the transmissive diffraction portion to the piezoelectric element to cover both the first surface and the second surface.

In the ultrasonic wave unit according to one aspect of the invention, the swelling body may include a first swelling body positioned between the ultrasonic wave generation surface and the first surface and configured to adhere the transmissive diffraction portion to the piezoelectric element, and a second swelling body attachable to and detachable from the second surface.

In the ultrasonic wave unit according to one aspect of the invention, the swelling body may have a contact surface that is in contact with the ultrasonic wave generation surface, and an exposed surface that is a surface opposite to the contact surface and is exposed to an outside of the ultrasonic wave unit, in a direction from the contact surface toward the exposed surface, the swelling body positioned between the first surface and the contact surface may have a first thickness, and the swelling body positioned between the second surface and the exposed surface may have a second thickness, and by adjusting at least one of the first thickness and the second thickness, a focal length from the exposed surface to a focal point or a convergence rate of the ultrasonic wave may be adjusted.

In the ultrasonic wave unit according to one aspect of the invention, the swelling body may have a contact surface that is in contact with the ultrasonic wave generation surface, and an exposed surface that is a surface opposite to the contact surface and is exposed to an outside of the ultrasonic wave unit, the transmissive diffraction portion may have a slit, and by adjusting a width of the slit, a focal length from the exposed surface to a focal point or a convergence rate of the ultrasonic wave may be adjusted.

In the ultrasonic wave unit according to one aspect of the invention, the transmissive diffraction portion may include a first member, a second member away from the first member and surrounding the first member, and a coupling portion positioned between the first member and the second member and coupling the first member to the second member.

A diffraction swelling tape according to one aspect of the invention is used in the ultrasonic wave unit according to the above aspect. The diffraction swelling tape includes a transmissive diffraction portion having a first surface and a second surface opposite to the first surface, and a swelling body covering at least one of the first surface and the second surface and adhered to a piezoelectric element.

An ultrasonic wave focusing device according to one aspect of the invention includes: an ultrasonic wave unit; a signal generation unit configured to supply a frequency signal to the ultrasonic wave unit; and an AC voltage generation unit configured to supply an AC voltage to the signal generation unit, in which the ultrasonic wave unit includes a piezoelectric element having an ultrasonic wave generation surface for generating an ultrasonic wave, and a transmissive diffraction portion positioned on the ultrasonic wave generation surface or away from the ultrasonic wave generation surface.

### Advantageous Effects of Invention

According to the ultrasonic wave unit, the diffraction swelling tape, and the ultrasonic wave focusing device according to aspects of the invention, a processing cost can be reduced by having a structure that can be easily processed.

### BRIEF DESCRIPTION OF DRAWINGS

FIG. 1 is a functional block diagram illustrating an example of a configuration of an ultrasonic wave focusing device according to a first embodiment of the invention.

FIG. 2 is a schematic cross-sectional view partially showing a structure of an ultrasonic wave unit according to the first embodiment of the invention, as viewed from a direction parallel to the ultrasonic wave unit.

FIG. 3A is a schematic cross-sectional view partially showing the structure of the ultrasonic wave unit according to the first embodiment of the invention, as viewed from a thickness direction of the ultrasonic wave unit.

FIG. 3B is a schematic cross-sectional view partially showing a modification of an FZP member constituting the ultrasonic wave unit according to the first embodiment of the invention, as viewed from the thickness direction of the ultrasonic wave unit.

FIG. 4 is a schematic cross-sectional view partially showing a structure of an ultrasonic wave unit according to a second embodiment of the invention, as viewed from a direction parallel to the ultrasonic wave unit.

FIG. 5 is a schematic cross-sectional view partially showing a structure of an ultrasonic wave unit according to a third embodiment of the invention, as viewed from a direction parallel to the ultrasonic wave unit.

FIG. 6 is a schematic cross-sectional view partially showing a structure of an ultrasonic wave unit according to a fourth embodiment of the invention, as viewed from a direction parallel to the ultrasonic wave unit.

FIG. 7 is a schematic cross-sectional view partially showing a structure of an ultrasonic wave unit according to a fifth embodiment of the invention, as viewed from a direction parallel to the ultrasonic wave unit.

FIG. 8A is a schematic cross-sectional view partially showing a structure of a diffraction gel tape according to a sixth embodiment of the invention, as viewed from a direction parallel to the diffraction gel tape.

FIG. 8B is a plan view partially showing a modification of the diffraction gel tape according to the sixth embodiment of the invention.

FIG. 9 is a schematic cross-sectional view partially showing a structure of a diffraction gel tape according to a seventh embodiment of the invention, as viewed from a direction parallel to the diffraction gel tape.

FIG. 10A is a schematic cross-sectional view partially showing a structure of a diffraction gel tape according to the seventh embodiment of the invention, and is a view illustrating a method for manufacturing the diffraction gel tape.

FIG. 10B is a schematic cross-sectional view partially showing the structure of the diffraction gel tape according to the seventh embodiment of the invention, and is a view illustrating the method for manufacturing the diffraction gel tape.

FIG. 11A is a schematic cross-sectional view partially showing the structure of the diffraction gel tape according to a modification of the seventh embodiment of the invention, and is a view illustrating a method for manufacturing the diffraction gel tape.

FIG. 11B is a schematic cross-sectional view partially showing the structure of the diffraction gel tape according to the modification of the seventh embodiment of the invention, and is a view illustrating the method for manufacturing the diffraction gel tape.

FIG. 11C is a schematic cross-sectional view partially showing the structure of the diffraction gel tape according to the modification of the seventh embodiment of the invention, and is a view illustrating the method for manufacturing the diffraction gel tape.

FIG. 12A is a cross-sectional view schematically showing an ultrasonic wave unit according to an example of the invention, and is a view illustrating conditions of simulation.

FIG. 12B is a cross-sectional view schematically showing an ultrasonic wave unit according to Example 1 of the invention, and is a view illustrating an analysis model for the simulation.

FIG. 12C is a diagram showing results of the simulation of the ultrasonic wave unit according to Example 1 of the invention.

FIG. 12D is a diagram showing results of the simulation of the ultrasonic wave unit according to Example 1 of the invention.

FIG. 12E is a diagram showing results of the simulation of the ultrasonic wave unit according to Example 1 of the invention.

FIG. 13A is a cross-sectional view schematically showing an ultrasonic wave unit of Comparative Example, and is a view illustrating conditions of simulation.

FIG. 13B is a cross-sectional view schematically showing the ultrasonic wave unit of Comparative Example, and is a view illustrating an analysis model for the simulation.

FIG. 13C is a diagram showing results of the simulation of the ultrasonic wave unit of Comparative Example.

FIG. 13D is a diagram showing results of the simulation of the ultrasonic wave unit of Comparative Example.

FIG. 14A is a diagram showing results of simulation of an ultrasonic wave unit according to Example 2 of the invention.

FIG. 14B is a diagram showing results of the simulation of the ultrasonic wave unit according to Example 2 of the invention.

FIG. 14C is a diagram showing results of the simulation of the ultrasonic wave unit according to Example 2 of the invention.

FIG. 14D is a diagram showing results of the simulation of the ultrasonic wave unit according to Example 2 of the invention.

FIG. 15 is a diagram showing results of comparison between the invention and Comparative Example in regard of a maximum ultrasonic wave intensity, relating to Example 3 of the invention.

FIG. 16 is a table showing conditions of Example 4 of the invention.

FIG. 17 is a diagram showing a configuration of a test device used in Example 4 of the invention.

FIG. 18 is a diagram showing results of simulation for Comparative Example relating to Example 4 of the invention.

FIG. 19 is a diagram showing experimental results of Comparative Example using the test device relating to Example 4 of the invention.

FIG. 20 is a diagram showing results of simulation for a lens 1 relating to Example 4 of the invention.

FIG. 21 is a diagram showing experimental results of the lens 1 using the test device relating to Example 4 of the invention.

FIG. 22 is a diagram showing results of simulation for a lens 2 relating to Example 4 of the invention.

FIG. 23 is a diagram showing experimental results of the lens 2 using the test device relating to Example 4 of the invention.

FIG. 24 is a diagram showing results of simulation for Comparative Example relating to Example 4 of the invention.

FIG. 25 is a diagram showing experimental results of Comparative Example using the test device relating to Example 4 of the invention.

FIG. 26 is a diagram showing results of simulation for a lens 3 relating to Example 4 of the invention.

FIG. 27 is a diagram showing experimental results of the lens 3 using the test device relating to Example 4 of the invention.

FIG. 28 is a diagram showing results of simulation for a lens 4 relating to Example 4 of the invention.

FIG. 29 is a diagram showing experimental results of the lens 4 using the test device relating to Example 4 of the invention.

FIG. 30 is a table summarizing the results of simulation and the experimental results for the lenses 1 to 4 relating to Example 4 of the invention.

### DESCRIPTION OF EMBODIMENTS

An ultrasonic wave unit, a diffraction swelling tape, and an ultrasonic wave focusing device according to embodiments of the invention will be described with reference to the drawings.

In the description of the embodiments, components having the same or similar functions are denoted by the same reference numerals. Redundant descriptions of the configurations may be omitted. The drawings are schematic or conceptual, and a relation between thickness and width of each portion, a ratio of sizes between portions, and the like are not necessarily the same as actual ones.

In the description of the embodiments, ordinal numbers such as “first” and “second” may be used. The ordinal numbers do not indicate the number of members described in the ordinal numbers. The ordinal numbers may be used to indicate that each of the plurality of members is a separate member.

In the drawings referred to in the description of the embodiments, three directions corresponding to a three-dimensional orthogonal coordinate system, that is, an X direction, a Y direction, and a Z direction are illustrated (reference numerals X, Y, and Z). The Z direction corresponds to a thickness direction of the ultrasonic wave unit, or corresponds to a direction from a contact surface of a gel member toward an exposed surface. In other words, the Z direction corresponds to a direction in which an ultrasonic wave generated from a piezoelectric element propagates toward a swelling body. One of two directions perpendicular to the Z direction is referred to as the X direction, and the other is referred to as the Y direction. The X direction and the Y direction may be referred to as directions parallel to the ultrasonic wave unit, or directions parallel to a diffraction gel tape. A direction extending from a center of the ultrasonic wave unit in a radial pattern as viewed in the Z direction is referred to as a radial direction. When viewed in the Z direction, a circumferential direction of the ultrasonic wave unit having a circular shape is referred to as a circumferential direction.

However, the wording “X direction”, “Y direction”, and “Z direction” are wording used to explain mutual positional relations among a plurality of members constituting the ultrasonic wave unit and the shape and structure of each of the plurality of members, and does not define a posture of the ultrasonic wave unit. According to a use state of the ultrasonic wave unit, the posture of the ultrasonic wave unit is freely changed. In such a use state, for example, the ultrasonic wave unit may be reversed or inclined in one direction among the three directions.

In the following embodiments, the ultrasonic wave unit, the diffraction gel tape, and the ultrasonic wave focusing device will be described under a case of being applied to, for example, an acupuncture point stimulation device. According to such an acupuncture point stimulation device, by using a function of ultrasonic wave focusing, it is possible to apply a stimulus to a living body such as a human body or an animal non-invasively (without damaging the living body) from a skin surface of the living body to a treatment site (focal point) positioned at a desired depth. Therefore, according to such ultrasonic wave stimulation, the same effect as needle stimulation can be obtained.

### First Embodiment

### <Structure of Ultrasonic Wave Focusing Device>

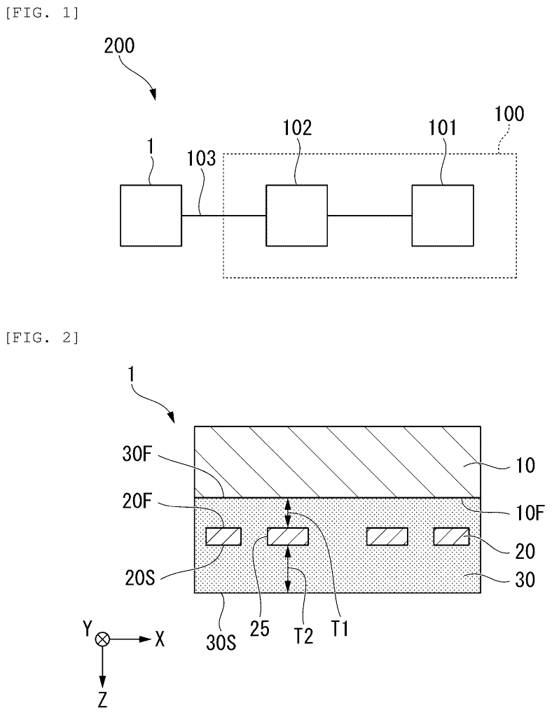

FIG. 1 is a functional block diagram showing an example of a configuration of an ultrasonic wave focusing device 200.

The ultrasonic wave focusing device 200 includes an ultrasonic wave unit 1, a device body 100, and a signal cable 103. The device body 100 includes an AC voltage generation unit 101 and a signal generation unit 102. The ultrasonic wave unit 1 is electrically connected to the signal generation unit 102 of the device body 100 via the signal cable 103. The ultrasonic wave unit 1 generates an ultrasonic wave based on a frequency signal received from the signal generation unit 102.

When the ultrasonic wave focusing device 200 is used, the ultrasonic wave focusing device 200 is connected to an external power supply via a power cable (not shown). The external power supply is, for example, a commercial power supply that supplies power to a power outlet. Electric power of the external power supply is, for example, AC power such as AC 100 V. In this case, the ultrasonic wave focusing device 200 using an external power supply can be implemented.

Note that instead of the structure using the external power supply, the ultrasonic wave focusing device 200 may include a chargeable battery. In this case, it is not necessary to connect the external power supply and the ultrasonic wave focusing device 200, and a portable ultrasonic wave focusing device 200 using electric power charged in the battery can be implemented.

In the following description, the ultrasonic wave focusing device 200 using an external power supply will be described.

### <AC Voltage Generation Unit 101>

The AC voltage generation unit 101 is connected to the signal generation unit 102 via a wiring embedded in the ultrasonic wave focusing device 200. The AC voltage generation unit 101 is configured to supply an AC voltage to the signal generation unit 102. The AC voltage generation unit 101 is a circuit configured to boost the AC power supplied from the external power supply to the ultrasonic wave focusing device 200. The AC voltage generation unit 101 includes, for example, a transformer and an inverter. A circuit configuration of the AC voltage generation unit 101 is not limited as long as an AC voltage can be supplied to the signal generation unit 102, and a known circuit may be used. The AC voltage generation unit 101 may also be referred to as a power amplifier.

### <Signal Generation Unit 102>

The signal generation unit 102 is connected to the ultrasonic wave unit 1 via the signal cable 103. The signal generation unit 102 is a circuit configured to supply a frequency signal to the ultrasonic wave unit 1. Regarding the frequency signal, a numerical value, a signal waveform, a voltage value, and the like of the frequency are not particularly limited as long as effects of the ultrasonic wave unit 1 can be sufficiently obtained.

As the frequency signal, for example, a continuous wave having a frequency corresponding to a design value of an FZP member 20 to be described later is used. Such a continuous wave is, for example, a sine wave or a burst wave. The frequency signal supplied from the signal generation unit 102 to the ultrasonic wave unit 1 is not limited to a sine wave or a burst wave, and signals having other waveforms may be used. For example, a signal having a waveform of an ultrasonic wave pulse may be used.

A circuit configuration of the signal generation unit 102 is not limited as long as a continuous wave as the frequency signal can be supplied to the ultrasonic wave unit 1, and a known circuit may be used. The signal generation unit 102 may also be referred to as an AC waveform generation unit.

### <Ultrasonic Wave Unit 1>

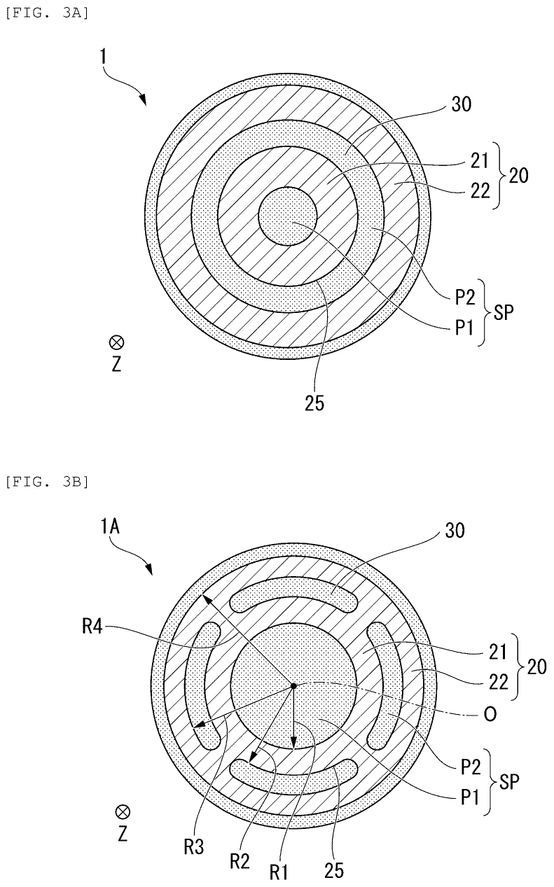

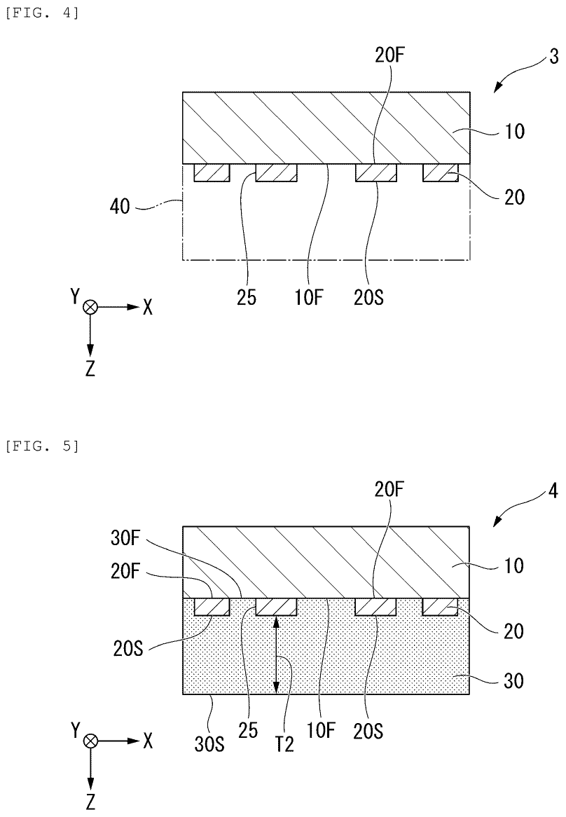

FIG. 2 is a schematic cross-sectional view partially illustrating a structure of the ultrasonic wave unit 1 according to the first embodiment, as viewed from a direction parallel to the ultrasonic wave unit 1. FIG. 3A is a schematic cross-sectional view partially showing the structure of the ultrasonic wave unit 1 according to the first embodiment, as viewed from a thickness direction of the ultrasonic wave unit 1.

As shown in FIGS. 2 and 3A, the ultrasonic wave unit 1 includes a piezoelectric element 10, a Fresnel zone plate (FZP) member 20, and a gel member 30.

The FZP member 20 is an example of a transmissive diffraction portion.

The gel member 30 is an example of a swelling body.

The ultrasonic wave unit 1 has a circular shape as viewed in the Z direction. Note that the shape of the ultrasonic wave unit 1 may be a shape other than a circle.

A width of the ultrasonic wave unit 1 in the X direction and the Y direction, that is, a diameter of the ultrasonic wave unit 1 is, for example, approximately 5 mm to 6 mm. However, the present embodiment does not limit the diameter of the ultrasonic wave unit 1.

For a length of the ultrasonic wave unit 1 in the Z direction, that is, a thickness of the ultrasonic wave unit 1, the thickness of the ultrasonic wave unit 1 can be appropriately changed depending on the thickness of the FZP member 20 and the gel member 30 constituting the ultrasonic wave unit 1.

In the example shown in FIGS. 2 and 3A, the ultrasonic wave unit 1 includes the gel member 30, but the ultrasonic wave unit 1 does not necessarily need to include the gel member 30. That is, the ultrasonic wave unit 1 only needs to include the piezoelectric element 10 and the FZP member 20 at least.

Regarding a positional relation between the piezoelectric element 10 and the FZP member 20 in the ultrasonic wave unit 1, in the structure shown in FIG. 2, the piezoelectric element 10 and the FZP member 20 are away from each other, but the piezoelectric element 10 does not have to be away from the FZP member 20. In other words, the FZP member 20 may be positioned on the piezoelectric element 10 so as to be in contact with the piezoelectric element 10. In the example illustrated in FIGS. 2 and 3A, the FZP member 20 is formed of a member different from that of the piezoelectric element 10.

### <Piezoelectric Element 10>

The piezoelectric element 10 is a passive element using a piezoelectric effect. Specifically, the piezoelectric element 10 includes an upper electrode, a lower electrode, and a piezoelectric body sandwiched between the upper electrode and the lower electrode. A voltage of the frequency signal supplied from the signal generation unit 102 to the piezoelectric element 10 is applied to the upper electrode and the lower electrode. The voltage of the frequency signal applied between the upper electrode and the lower electrode is applied to the piezoelectric body, and the piezoelectric body vibrates. That is, the piezoelectric element 10 is configured to convert a voltage signal applied to the piezoelectric element 10 into a vibration motion.

For example, the piezoelectric body is formed using a ferroelectric ceramic such as lead (Pb) zirconate titanate (PZT). A material constituting the piezoelectric body is not limited to PZT, and other piezoelectric element materials may also be adopted.

A structure of the piezoelectric element 10 is not particularly limited. The piezoelectric element 10 may include, for example, a lower electrode, an upper electrode, and a support substrate that supports the piezoelectric body. The support substrate may have a wiring pattern connected to each of the lower electrode and the upper electrode. In this case, the signal cable 103, the lower electrode, and the upper electrode are connected to each other via the wiring pattern formed on the support substrate.

In the examples shown in FIGS. 2 and 3A, the piezoelectric element 10 has the same shape as that of the ultrasonic wave unit 1 when viewed in the Z direction, but the shape of the piezoelectric element 10 is not particularly limited. From the viewpoint of reducing a manufacturing cost of the ultrasonic wave unit 1, the piezoelectric element 10 is preferably a commercially available product in the related art.

The piezoelectric element 10 has an ultrasonic wave generation surface 10F that generates an ultrasonic wave. The ultrasonic wave generation surface 10F is, for example, a surface of one of the upper electrode and the lower electrode constituting the piezoelectric element 10. Note that the ultrasonic wave generation surface 10F may not be the surface of one of the upper electrode and the lower electrode. As long as the vibration of the piezoelectric element 10 can be transmitted to the ultrasonic wave generation surface 10F and the ultrasonic wave generation surface 10F can generate the ultrasonic wave, the position of the ultrasonic wave generation surface 10F in the structure of the piezoelectric element 10 is not limited. For example, the ultrasonic wave generation surface 10F may be formed at a site inside an electrode formed in a ring shape when viewed in the Z direction.

In the present embodiment, the ultrasonic wave generation surface 10F is a surface that comes into contact with the gel member 30, and is a surface on which the gel member 30 is peeled off. The ultrasonic wave generation surface 10F of the piezoelectric element 10 which is a commercially available product is, for example, a planar surface. The structure and the shape of the ultrasonic wave generation surface 10F are not particularly limited. A surface of the ultrasonic wave generation surface 10F may be an uneven surface in which an adhesion force between the ultrasonic wave generation surface 10F and the gel member 30 and a peeling property of the gel member 30 from the ultrasonic wave generation surface 10F are considered. In this case, for example, the ultrasonic wave generation surface 10F may have an uneven surface on which fine unevenness is formed. In other words, the ultrasonic wave generation surface 10F may have surface roughness in consideration of the adhesion force and the peeling property with respect to the gel member 30.

Note that the shape of the ultrasonic wave generation surface 10F of the piezoelectric element 10 is not limited to a planar shape, and may have a curved surface, unevenness, or the like. The piezoelectric element 10 may be a flexible piezoelectric element. As the flexible piezoelectric element, for example, a structure using a flexible film can be adopted.

### <FZP Member 20>

The FZP member 20 is a planar member having a slit 25.

The FZP member 20 is an example of an acoustic lens.

In the present embodiment, the FZP member 20 includes an inner FZP portion 21 and an outer FZP portion 22. The outer FZP portion 22 is in a position surrounding the inner FZP portion 21. The inner FZP portion 21 and the outer FZP portion 22 are away from each other. The slit 25 is formed between the inner FZP portion 21 and the outer FZP portion 22.

The inner FZP portion 21 is an example of a first member.

The outer FZP portion 22 is an example of a second member.

In other words, the FZP member 20 has a concentric circular slit pattern SP formed in a planar member. Specifically, the slit pattern SP has a center opening P1 and a peripheral opening P2. The center opening P1 has a circular shape. The peripheral opening P2 has an annular shape. The peripheral opening P2 is in a position surrounding the center opening P1. The center opening P1 and the peripheral opening P2 are concentric. The FZP member 20 is a member that causes diffraction of the ultrasonic wave by the concentric circular slit pattern SP.

The FZP member 20 has a function as a lens that generates a diffraction phenomenon (Fresnel diffraction) by generating interference of ultrasonic waves and generates interference fringes of ultrasonic waves at a focal point. Therefore, the FZP member 20 may also be referred to as a diffraction member, a diffraction lens, an FZP lens, a Fresnel lens, or the like.

In the ultrasonic wave unit 1 using the FZP member 20, a focal length can be adjusted depending on a frequency of the ultrasonic wave.

In other words, the configuration of the FZP member 20 can be adjusted according to the frequency signal supplied from the ultrasonic wave focusing device 200 to the piezoelectric element 10 and the frequency of the ultrasonic wave generated from the piezoelectric element 10.

In the following description, the wording “focal point” is a position where the ultrasonic wave is focused. In other words, the focal point corresponds to a treatment site in a depth direction from the skin of the living body.

The wording “focal length” corresponds to a distance from an end in the Z direction of the ultrasonic wave unit 1 to the focal point. In other words, the wording “focal length” corresponds to a distance between an exposed surface 30S of the gel member 30 and the focal point, that is, a distance between the surface of the skin of the living body and the treatment site.

As a material of the planar member constituting the FZP member 20, for example, acrylic resin (PMMA, polymethyl methacrylate) is used. Note that materials other than the acrylic resin may be used as the material of the FZP member 20.

A thickness of the FZP member 20 in the Z direction is substantially equal to a wavelength of the ultrasonic wave propagating through the gel member 30. A wavelength A is obtained by dividing a propagation speed v by a frequency f(λ=v/f). The propagation speed of the ultrasonic wave propagating through the gel member 30 used in the present embodiment is 1141.5 m/s. In this case, when the frequency of the ultrasonic wave is 2 MHZ, the wavelength of the ultrasonic waves is 570.75 μm, and the thickness of the FZP member 20 is set to approximately 0.6 mm. When the frequency of the ultrasonic wave is 3 MHZ, the wavelength of the ultrasonic wave is 380.5 μm, and the thickness of the FZP member 20 is set to approximately 0.4 mm.

The FZP member 20 is positioned away from the ultrasonic wave generation surface 10F. Specifically, the FZP member 20 includes a first FZP surface 20F facing and away from the ultrasonic wave generation surface 10F and a second FZP surface 20S opposite to the first FZP surface 20F.

The first FZP surface 20F is an example of a first surface.

The second FZP surface 20S is an example of a second surface.

The slit 25 of the FZP member 20 is a site where the ultrasonic wave generated from the piezoelectric element 10 transmits. In other words, the ultrasonic wave does not transmit through a site of the FZP member 20 where the slit 25 is not formed. A width of the slit 25 in a radial direction is determined according to the focal length or a convergence rate of the ultrasonic wave when the ultrasonic wave unit 1 is used. The width of the slit 25 is selected, for example, within a range of 0.4 mm to 1.0 mm. However, the present embodiment does not limit the width of the slit 25.

As a method for forming the slit 25 in the planar member serving as a mother material of the FZP member 20, for example, laser processing, cutting processing, etching, or the like is adopted. High accuracy is not required for processing the slit 25 as long as a target width of the slit 25 can be ensured.

In the structure shown in FIG. 2, the FZP member 20 is disposed away from the ultrasonic wave generation surface 10F, but the FZP member 20 does not have to be away from the ultrasonic wave generation surface 10F. In other words, the FZP member 20 may be in contact with the ultrasonic wave generation surface 10F. That is, the FZP member 20 may be positioned on the ultrasonic wave generation surface 10F or may be positioned away from the ultrasonic wave generation surface 10F.

### <Modification 1 of FZP Member 20>

FIG. 3B is a schematic cross-sectional view partially showing a modification of the FZP member 20 constituting an ultrasonic wave unit 1A according to the first embodiment, as viewed from a thickness direction of the ultrasonic wave unit 1A.

The FZP member 20 shown in FIG. 3B is different from the FZP member 20 shown in FIG. 3A in that a coupling portion is provided between the inner FZP portion 21 and the outer FZP portion 22.

As shown in FIG. 3B, the FZP member 20 includes a coupling portion 23 that couples the inner FZP portion 21 to the outer FZP portion 22. The coupling portion 23 extends in the radial direction, for example. The coupling portion 23 may extend in a direction inclined in the radial direction. In the example shown in FIG. 3B, the number of the coupling portions 23 is four, but the number of the coupling portions 23 may be one to three or five or more. For example, the number of the coupling portions 23 can be determined according to strength required for the FZP member 20.

In the example shown in FIG. 3B, the slit pattern SP includes one center opening P1 and a plurality of peripheral openings P2. Since the number of the coupling portions 23 is four, the number of the peripheral openings P2 is also four. The four peripheral openings P2 are arranged along the circumferential direction. Each of the four peripheral openings P2 is a curved long hole. The long hole has a curved shape extending along the circumferential direction. The four peripheral openings P2 are positioned surrounding the center opening P1. The center opening P1 and the four peripheral openings P2 are concentric.

Two of the peripheral openings P2 adjacent to each other are away from each other in the circumferential direction. In other words, the four peripheral openings P2 may be referred to as annular shapes having separate portions formed by the coupling portions 23.

Since the inner FZP portion 21 and the outer FZP portion 22 are coupled by the coupling portions 23, the inner FZP portion 21 and the outer FZP portion 22 can be integrated. In this way, the strength of the FZP member 20 can be improved. Further, a distance between the inner FZP portion 21 and the outer FZP portion 22, that is, the width of the slit 25 can be maintained constant by the coupling portions 23.

### <Modification 2 of FZP Member 20>

In the examples shown in FIGS. 3A and 3B, the slit pattern SP in the FZP member 20 is a concentric circle pattern, but the slit pattern SP is not limited to a concentric circular pattern. The FZP member 20 may have a slit pattern SP that extends linearly in the X direction, in the Y direction, or in an oblique direction that is obliquely inclined with respect to the X direction. That is, the FZP member 20 may include a diffraction grating having a line-and-space pattern. Further, a spiral pattern may be adopted as the slit pattern SP in the FZP member 20.

### <Modification 3 of FZP Member 20>

In the examples shown in FIGS. 3A and 3B, the shape of the center opening P1 is circular, but other shapes may be adopted as the shape of the center opening P1. For example, the center opening P1 may have a long hole shape or an elliptical shape.

An annular shape formed by one or a plurality of peripheral openings P2 may be changed according to the shape of the center opening P1 as long as a certain slit width can be obtained.

The FZP member 20 may have a hologram element, in other words, a holographic diffraction grating, as long as the FZP member 20 can generate the effect of the diffraction interference of the ultrasonic wave.

The FZP member 20 may perform beam forming of the ultrasonic wave, collimation of the ultrasonic wave, and modulation of the ultrasonic wave.

<Selection from Plurality of FZP Members>

A plurality of types of FZP members having different widths of the slit 25, that is, N types of FZP members (N is an integer of 2 or more) may be prepared.

Specifically, first, a plurality of types of FZP members set according to the type of a treatment target, the type of the treatment site, the type of the focal length, or the type of the convergence rate of the ultrasonic wave are designed. In the design of the plurality of types of FZP members, the width of the slit 25 is freely selected, and the width of the slit 25 is determined according to the type of the treatment target, the type of the treatment site, the type of the focal length, or the type of the convergence rate of the ultrasonic wave by performing an experiment or a simulation relating to each slit 25.

In other words, by performing an experiment or a simulation while adjusting the width of the slit 25, it is possible to determine the slit 25 according to the treatment target, the treatment site, the focal length, or the convergence rate of the ultrasonic wave.

According to the preparation described above, the plurality of types of FZP members having different widths of the slits 25 are obtained. The wording “different widths of the slits 25” means that the widths of the slits 25 are different from each other in the plurality of types of FZP members.

Note that in the design of the FZP members, not only the width of the slit 25 but also the slit pattern SP may be designed. Different slit patterns SP may be designed.

A method for selecting an FZP member having an appropriate slit 25 among a plurality of FZP members is as follows.

Here, the “FZP member having an appropriate slit 25” refers to an FZP member used in actual treatment, which is an FZP member selected by a user of the ultrasonic wave unit 1 taking into consideration an intended treatment target, an intended treatment site, an intended focal length, or an intended convergence rate of the ultrasonic wave.

The user determines the intended treatment target, the intended focal length, or the intended convergence rate of the ultrasonic wave. Next, the user selects an FZP member having the appropriate slit 25 among the plurality of types of FZP members according to the intended treatment target, the intended focal length, or the intended convergence rate of the ultrasonic wave. The selected FZP member is applied to the ultrasonic wave unit 1.

Therefore, by selecting an appropriate FZP member from the plurality of FZP members, it is possible to adjust the focal length or the convergence rate of the ultrasonic wave in the ultrasonic wave unit 1. In other words, the focal length or the convergence rate of the ultrasonic wave can be adjusted by adjusting the width of the slit 25.

### <Gel Member 30>

The gel member 30 is a member in a gel state containing a solid polymer and a solvent such as water. In other words, the gel member 30 is a member capable of elastic deformation. The gel member 30 has a structure in which a volume of the solid polymer is increased by absorbing the solvent.

As a material of the planar member constituting the gel member 30, for example, a urethane resin is used. A density of the gel member 30 is approximately 998.6 kg/m³. Note that materials other than the urethane resin may be used as the material of the gel member 30. The material of the gel member 30 preferably has excellent affinity with the skin of the living body.

As the material of the gel member 30, it is preferable to use a super-soft urethane resin having the same softness after curing as the softness of human skin. A method for curing the gel member 30 is not particularly limited. The curing may be performed by using a two-liquid mixed type material and flowing the material into a mold prepared in advance.

The gel member 30 can propagate the ultrasonic waves generated from the piezoelectric element 10 toward an outside of the ultrasonic wave unit 1. Therefore, the gel member 30 may also be referred to as a sound wave transmission gel.

The gel member 30 includes a contact surface 30F which is in contact with the ultrasonic wave generation surface 10F, and the exposed surface 30S which is a surface opposite to the contact surface 30F and is exposed to the outside of the ultrasonic wave unit 1.

The gel member 30 covers both the first FZP surface 20F and the second FZP surface 20S of the FZP member 20. The gel member 30 is provided on the ultrasonic wave generation surface 10F so as to cover the FZP member 20. In other words, the FZP member 20 is embedded in the gel member 30. In this way, the gel member 30 is integrated with the FZP member 20.

That is, the gel member 30 is disposed between the first FZP surface 20F and the ultrasonic wave generation surface 10F and is also disposed on the second FZP surface 20S. Further, the gel member 30 is also disposed inside the slit 25.

Since the cured gel member 30 is not a fluid, the position of the FZP member 20 inside the gel member 30 is stably maintained.

The gel member 30 has adhesiveness. Therefore, the gel member 30 can also be referred to as an adhesive gel.

Therefore, the contact surface 30F of the gel member 30 can be directly and easily attached to the ultrasonic wave generation surface 10F. In other words, the gel member 30 adheres at least the FZP member 20 to the piezoelectric element 10.

The gel member 30 can be easily peeled off from the ultrasonic wave generation surface 10F. In other words, an integrated body in which the gel member 30 and the FZP member 20 are integrated is attachable to and detachable from the piezoelectric element 10, and it is possible to dispose of only the integrated body without discarding the piezoelectric element 10.

In the present embodiment, the ultrasonic wave unit 1 is applied to an acupuncture point stimulation device. Therefore, the gel member 30 can be attached to the skin of the living body. That is, the exposed surface 30S of the gel member 30 is a surface to be attached to the skin of the living body. Therefore, the gel member 30 can also be referred to as a gel pad.

In the gel member 30 having such a configuration, intensity and the focal length of the ultrasonic wave can be adjusted by adjusting a thickness of the gel member 30. Furthermore, as described later, it is possible to more efficiently focus the ultrasonic wave than in the case of an acoustic lens having a concave surface.

Specifically, the gel member 30 has a first thickness T1 corresponding to a distance between the first FZP surface 20F and the contact surface 30F in the Z direction. Furthermore, the gel member 30 has a second thickness T2 corresponding to a distance between the second FZP surface 20S and the exposed surface 30S. By adjusting at least one of the first thickness T1 and the second thickness T2, the gel member 30 can adjust the focal length from the exposed surface 30S to the focal point or the convergence rate of the ultrasonic wave.

<Selection from Plurality of Gel Members>

A plurality of types of gel members having different thickness conditions, that is, N types of gel members (N is an integer of 2 or more) may be prepared. Here, the thickness condition means the first thickness T1 and the second thickness T2.

Specifically, first, a plurality of types of gel members set according to the type of the treatment target, the type of the treatment site, the type of the focal length, or the type of the convergence rate of the ultrasonic wave are designed. In the design of the plurality of types of gel members, at least one of the first thickness T1 and the second thickness T2 is freely selected, that is, the thickness condition is freely selected. By performing an experiment or a simulation relating to the gel member in accordance with the selected condition, the thickness condition corresponding to the type of the treatment target, the type of the treatment site, the type of the focal length, or the type of the convergence rate of the ultrasonic wave is determined.

In other words, by performing an experiment or a simulation while adjusting the thickness condition, it is possible to determine the thickness condition according to the treatment target, the treatment site, the focal length, or the convergence rate of the ultrasonic wave.

A plurality of types of gel members having different thickness conditions are obtained from the preparation described above. The wording “different thickness conditions” means that the thickness conditions are different from each other in the plurality of types of gel members. More specifically, for example, when two gel members have different thickness conditions, it means that at least one of the first thickness T1 and the second thickness T2 is different between the two gel members. In other words, it can be said that the thickness condition is different even when the first thickness T1 is the same and the second thickness T2 is different between the two gel members.

A method for selecting a gel member having an appropriate thickness condition among a plurality of gel members is as follows.

Here, the “gel member having an appropriate thickness condition” refers to a gel member used in actual treatment, which is a gel member selected by the user of the ultrasonic wave unit 1 taking into consideration the intended treatment target, the intended treatment site, the intended focal length, or the intended convergence rate of the ultrasonic wave.

The user determines the intended treatment target, the intended focal length, or the intended convergence rate of the ultrasonic wave. Next, the user selects a gel member having an appropriate thickness condition among the plurality of types of gel members according to the intended treatment target, the intended focal length, or the intended convergence rate of the ultrasonic wave. The selected gel member is applied to the ultrasonic wave unit 1.

Therefore, by selecting an appropriate gel member from the plurality of gel members, it is possible to adjust the focal length or the convergence rate of the ultrasonic wave in the ultrasonic wave unit 1. In other words, by adjusting at least one of the first thickness T1 and the second thickness T2, the focal length or the convergence rate of the ultrasonic waves can be adjusted.

### <Functions and Effects>

The user who uses the ultrasonic wave focusing device 200 brings the exposed surface 30S exposed to the outside of the ultrasonic wave unit 1 into contact with the skin of the living body. In this state, the ultrasonic wave focusing device 200 is driven.

Note that in a state where the ultrasonic wave focusing device 200 is driven, the exposed surface 30S may be brought into contact with the skin of the living body.

The ultrasonic wave focusing device 200 includes the ultrasonic wave unit 1, the AC voltage generation unit 101, the signal generation unit 102, and the signal cable 103. Therefore, the ultrasonic wave focusing device 200 can supply a frequency signal to the ultrasonic wave unit 1. When the ultrasonic wave unit 1 receives the frequency signal, a voltage of the frequency signal is applied to the piezoelectric element 10, and the piezoelectric element 10 vibrates. Accordingly, the piezoelectric element 10 can generate the ultrasonic wave from the ultrasonic wave generation surface 10F.

The ultrasonic wave propagates to the gel member 30 positioned between the ultrasonic wave generation surface 10F and the first FZP surface 20F and reaches the FZP member 20. The ultrasonic wave passes through the slit 25 and propagates to the gel member 30 positioned between the exposed surface 30S and the second FZP surface 20S. The ultrasonic wave reaches the skin of the living body and then reaches the treatment site at a position away from the skin surface of the living body. On the other hand, in a site where the slit 25 is not provided in the FZP member 20, the ultrasonic wave does not reach the skin of the living body.

In the ultrasonic wave passing through the slit 25 of the FZP member 20, ultrasonic wave interference occurs. Therefore, the FZP member 20 generates a diffraction phenomenon of the ultrasonic wave generated from the ultrasonic e generation surface 10F and focuses the ultrasonic wave at a focal point which is the treatment site. That is, the FZP member 20 can focus energy of the ultrasonic wave on the focal point. Thus, the FZP member 20 can give a stimulus to the treatment site.

According to the ultrasonic wave unit 1 of the present embodiment, the following excellent effects can be obtained.

In forming the FZP member 20, the slit 25 can be formed in the planar member without requiring high accuracy. Therefore, a step of forming the FZP member 20 is extremely simple. In other words, when an acoustic lens in the related art is formed, it is necessary to process a spherical surface having a highly accurate curvature into a concave surface, and thus the processing is highly difficult, making it difficult to reduce a processing cost.

On the other hand, according to the ultrasonic wave unit 1, the FZP member 20 functioning as an acoustic lens can be easily formed, and a processing cost of the ultrasonic wave unit 1 including the FZP member 20 can be reduced.

In an ultrasonic wave treatment device including an acoustic lens having a concave surface in the related art, not only a gel member but also an adhesive tape for attaching the acoustic lens to a piezoelectric element is required.

On the other hand, according to the ultrasonic wave unit 1, the ultrasonic wave unit 1 can be obtained simply by attaching the gel member 30 having adhesiveness to the ultrasonic wave generation surface 10F. Therefore, as compared with the structure in which the acoustic lens is attached to the piezoelectric element using an adhesive tape, the number of members constituting the ultrasonic wave unit 1 can be reduced. Therefore, the ultrasonic wave unit 1 can be easily manufactured, and the processing cost of the ultrasonic wave unit 1 including the FZP member 20 can be reduced.

Further, in the structure in which the adhesive tape is disposed between the acoustic lens and the piezoelectric element, an interface is generated between the acoustic lens and the adhesive tape, and an interface is generated between the piezoelectric element and the adhesive tape. That is, two interfaces are generated between the acoustic lens and the piezoelectric element. In such a structure in the related art, since attenuation of the ultrasonic wave occurs at the interfaces, an attenuation amount of the ultrasonic wave cannot be reduced.

In contrast, according to the ultrasonic wave unit 1, the gel member 30 having adhesiveness is attached to the ultrasonic wave generation surface 10F without using an adhesive tape. Therefore, since the number of interfaces is smaller than that in the structure in the related art, attenuation of the ultrasonic wave can be reduced.

In the ultrasonic wave treatment device including an acoustic lens having a concave surface in the related art, a gel member or a resin member may be embedded in the concave surface. In this case, when the gel or resin member is embedded in the concave surface, air may remain in the concave surface. When air remains in the concave surface, the ultrasonic wave may be attenuated.

In contrast, since the ultrasonic wave unit 1 including the FZP member 20 has a structure that does not use the concave surface, unlike the ultrasonic wave treatment device in the related art, air hardly enters the ultrasonic wave unit 1. Accordingly, it is possible to solve the problem that the ultrasonic wave is attenuated due to the air remaining in the concave surface.

In an ultrasonic wave treatment device including an acoustic lens in the related art, treatment is performed by putting the acoustic lens against the skin in a state where gel is applied on the skin of the living body.

In contrast, in the ultrasonic wave unit 1 including the gel member 30, the ultrasonic wave unit 1 can be used by bringing the exposed surface 30S of the gel member 30 into contact with the skin without applying gel onto the skin of the living body. Since the gel member 30 can be easily peeled off from the piezoelectric element 10, the used gel member 30 in contact with the skin can be peeled off from the piezoelectric element 10 and discarded after the treatment using the ultrasonic wave unit 1 is completed. By attaching a new gel member 30 to the piezoelectric element 10, the ultrasonic wave unit 1 can be used. Since the gel member 30 can be easily replaced, the ultrasonic wave unit 1 excellent in hygiene can be achieved.

Furthermore, in the case of the present embodiment, since it is not necessary to apply gel to the skin, it is not necessary to wipe the gel applied on the skin after using the ultrasonic wave unit 1, and it is not necessary to rub the skin for removing the gel. A treatment object will not feel uncomfortable.

The piezoelectric element 10 is a commercially available product in the related art. Therefore, it is not necessary to use a piezoelectric element having a special function or structure in the ultrasonic wave unit 1. The ultrasonic wave unit 1 can be implemented simply by attaching an integrated body in which the gel member 30 and the FZP member 20 are integrated to a piezoelectric element in the related art. Therefore, in an ultrasonic wave treatment device using an acoustic lens in the related art, the same effects as those of the ultrasonic wave unit 1 can be obtained by using the gel member 30 and the FZP member 20 instead of the acoustic lens in the related art.

### Other Embodiments

Next, other embodiments of the ultrasonic wave unit will be described.

In the embodiments described below, the same members as those of the first embodiment are denoted by the same reference numerals, and the description thereof is omitted or simplified. Configurations different from those described in the first embodiment will be mainly described. Regarding the omission of description, for example, the description related to the driving of the ultrasonic wave focusing device 200 used as an acupuncture point stimulation device is omitted.

As the FZP member 20 described in the following embodiment, an FZP member having no coupling portion 23 as shown in FIG. 3A may be used, or an FZP member having the coupling portion 23 as shown in FIG. 3B may be used.

### Second Embodiment

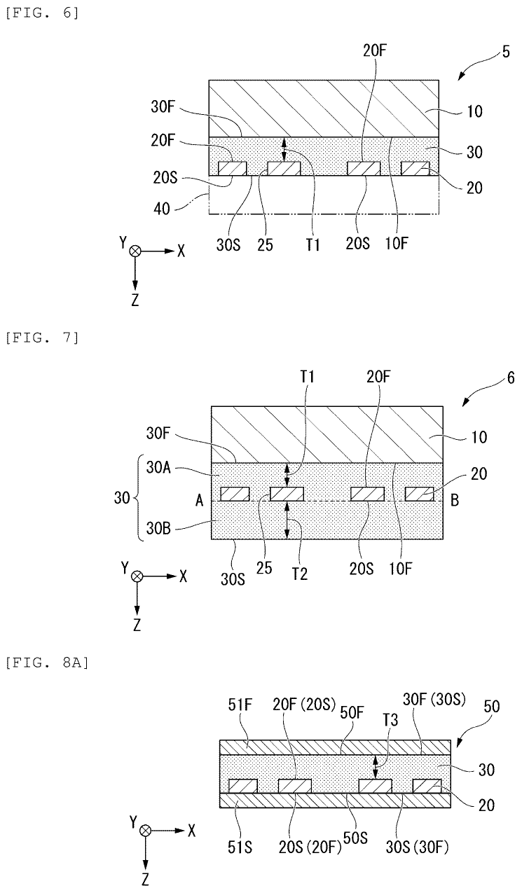

FIG. 4 is a schematic cross-sectional view partially showing a structure of an ultrasonic wave unit 3 according to a second embodiment, as viewed from a direction parallel to the ultrasonic wave unit 3.

The second embodiment is different from the first embodiment in that the FZP member is in contact with the ultrasonic wave generation surface of the piezoelectric element. Furthermore, in the second embodiment, the gel member 30 is not used.

As shown in FIG. 4, the ultrasonic wave unit 3 includes the piezoelectric element 10 and the FZP member 20 as in the first embodiment. The FZP member 20 is positioned on the ultrasonic wave generation surface 10F of the piezoelectric element 10. The FZP member 20 is positioned on the ultrasonic wave generation surface 10F. In other words, the ultrasonic wave generation surface 10F and the first FZP surface 20F are in contact with each other. No gel member is disposed between the ultrasonic wave generation surface 10F and the first FZP surface 20F. The FZP member 20 is a member different from the piezoelectric element 10.

A structure for fixing the FZP member 20 to the ultrasonic wave generation surface 10F is not particularly limited. For example, an adhesive may be disposed between the ultrasonic wave generation surface 10F and the FZP member 20, so that the FZP member 20 is fixed to the ultrasonic wave generation surface 10F. The FZP member 20 can be peeled off from the ultrasonic wave generation surface 10F.

In FIG. 4, a reference numeral 40 denotes an application gel which is disposed between the skin of the living body and the piezoelectric element 10 when the ultrasonic wave unit 3 is used. The application gel 40 may be applied to the skin of the living body first or may be applied to the ultrasonic wave generation surface 10F. In other words, the application gel 40 is not a component of the ultrasonic wave unit 3, but is a material used when the ultrasonic wave unit 3 is used. In the following description, the application gel 40 is applied to the skin of the living body.

### <Functions and Effects>

First, the application gel 40 is applied to the skin of the living body. The ultrasonic wave unit 3 is disposed on the skin via the application gel 40. Therefore, as shown in FIG. 4, the application gel 40 comes into contact with the ultrasonic wave generation surface 10F. The application gel 40 covers the whole of the ultrasonic wave generation surface 10F and the second FZP surface 20S so as to fill the slit 25 of the FZP member 20. In this state, the ultrasonic wave focusing device 200 is driven.

The ultrasonic wave generated from the ultrasonic wave generation surface 10F of the piezoelectric element 10 passes through the slit 25 and propagates to the application gel 40. The ultrasonic wave reaches the skin of the living body and then reaches the treatment site at a position away from the skin surface of the living body. On the other hand, in a site where the slit 25 is not provided in the FZP member 20, the ultrasonic wave does not reach the skin of the living body.

In the ultrasonic wave passing through the slit 25 of the FZP member 20, ultrasonic wave interference occurs. Therefore, the FZP member 20 generates a diffraction phenomenon of the ultrasonic wave generated from the ultrasonic wave generation surface 10F and focuses the ultrasonic wave at a focal point which is the treatment site. That is, the FZP member 20 can focus energy of the ultrasonic wave on the focal point. Thus, the FZP member 20 can give a stimulus to the treatment site.

According to the ultrasonic wave unit 3 of the present embodiment, unlike the first embodiment described above, the number of members constituting the ultrasonic wave unit 3 can be reduced by not using the FZP member 20. Therefore, the ultrasonic wave unit 3 can be easily manufactured.

### Third Embodiment

FIG. 5 is a schematic cross-sectional view partially showing a structure of an ultrasonic wave unit 4 according to a third embodiment, as viewed from a direction parallel to the ultrasonic wave unit 4.

The third embodiment is different from the first embodiment in that the FZP member is in contact with the ultrasonic wave generation surface of the piezoelectric element.

As shown in FIG. 5, the ultrasonic wave unit 4 includes the piezoelectric element 10, the FZP member 20, and the gel member 30 as in the first embodiment. The FZP member 20 is positioned on the ultrasonic wave generation surface 10F of the piezoelectric element 10. The FZP member 20 is positioned on the ultrasonic wave generation surface 10F. In other words, the ultrasonic wave generation surface 10F and the first FZP surface 20F are in contact with each other. The FZP member 20 is a member different from the piezoelectric element 10.

The gel member 30 covers the whole of the ultrasonic wave generation surface 10F and the second FZP surface 20S so as to fill the slit 25 of the FZP member 20. The gel member 30 is not disposed between the ultrasonic wave generation surface 10F and the first FZP surface 20F. In other words, the first thickness T1 of the gel member 30 is zero. The second thickness T2 of the gel member 30 can be appropriately changed according to the design of the gel member 30.

### <Functions and Effects>

First, the exposed surface 30S of the gel member 30 is brought into contact with the skin of the living body. In this state, the ultrasonic wave focusing device 200 is driven.

The ultrasonic wave generated from the ultrasonic wave generation surface 10F of the piezoelectric element 10 passes through the slit 25 and propagates to the gel member 30. The ultrasonic wave reaches the skin of the living body and then reaches the treatment site at a position away from the skin surface of the living body. On the other hand, in a site where the slit 25 is not provided in the FZP member 20, the ultrasonic wave does not reach the skin of the living body.

In the ultrasonic wave passing through the slit 25 of the FZP member 20, ultrasonic wave interference occurs. Therefore, the FZP member 20 generates a diffraction phenomenon of the ultrasonic wave generated from the ultrasonic wave generation surface 10F and focuses the ultrasonic wave at a focal point which is the treatment site. That is, the FZP member 20 can focus energy of the ultrasonic wave on the focal point. Thus, the FZP member 20 can give a stimulus to the treatment site.

According to the ultrasonic wave unit 4 of the present embodiment, unlike the first embodiment described above, the gel member 30 is not disposed between the ultrasonic wave generation surface 10F and the first FZP surface 20F, and thus the configuration of the gel member 30 can be simplified. Especially, it is not necessary to adjust the first thickness T1 of the gel member 30. By adjusting only the second thickness T2, the thickness of the gel member 30 can be adjusted. Therefore, it is easy to manage the thickness of the gel member 30.

### Fourth Embodiment

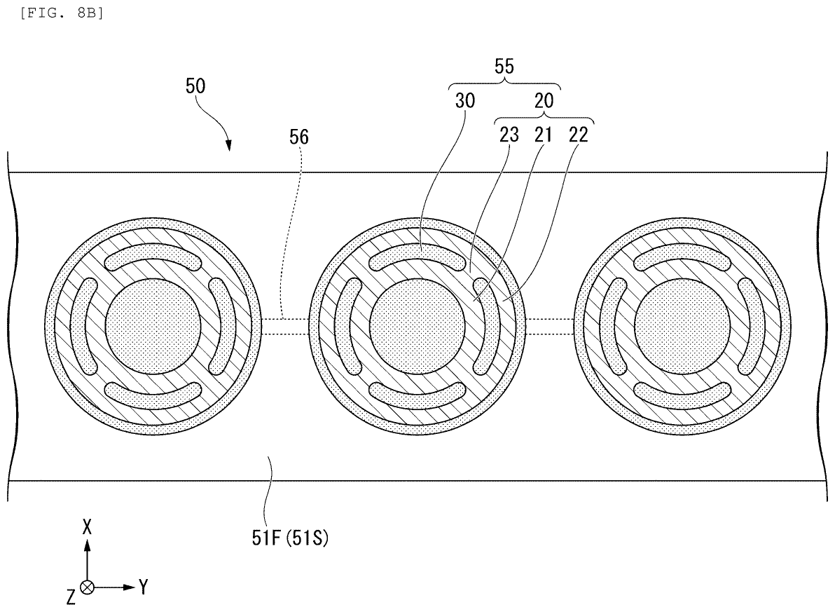

FIG. 6 is a schematic cross-sectional view partially showing a structure of an ultrasonic wave unit 5 according to a fourth embodiment, as viewed from a direction parallel to the ultrasonic wave unit 5.

The fourth embodiment is different from the first embodiment in the structure of the gel member 30.

As shown in FIG. 6, the ultrasonic wave unit 5 includes the piezoelectric element 10, the FZP member 20, and the gel member 30 as in the first embodiment. The FZP member 20 is positioned away from the ultrasonic wave generation surface 10F.

The gel member 30 covers the ultrasonic wave generation surface 10F and the first FZP surface 20F so as to fill the slit 25 of the FZP member 20. The second FZP surface 20S is not covered by the gel member 30. That is, in the Z direction, the position of the second FZP surface 20S and the position of the exposed surface 30S of the gel member 30 coincide with each other. In other words, the second thickness T2 of the gel member 30 is zero. The first thickness T1 of the gel member 30 is appropriately changed according to the design of the gel member 30.

The application gel 40 shown in FIG. 6 is the same as that in the second embodiment described above. The application gel 40 may be previously applied to the skin of the living body, or may be applied to the exposed surface 30S. In other words, the application gel 40 is not a component of the ultrasonic wave unit 5, but is a material used when the ultrasonic wave unit 5 is used. In the following description, the application gel 40 is applied to the skin of the living body.

### <Functions and Effects>

First, the application gel 40 is applied to the skin of the living body. The ultrasonic wave unit 5 is disposed on the skin via the application gel 40. In this way, as shown in FIG. 5, the application gel 40 covers the whole exposed surface 30S. In this state, the ultrasonic wave focusing device 200 is driven.

The ultrasonic wave generated from the ultrasonic wave generation surface 10F of the piezoelectric element 10 propagates to the gel member 30 positioned between the ultrasonic wave generation surface 10F and the first FZP surface 20F and reaches the FZP member 20. The ultrasonic wave passes through the slit 25 provided in the FZP member 20 and propagates to the application gel 40. The ultrasonic wave reaches the skin of the living body and then reaches the treatment site at a position away from the skin surface of the living body. On the other hand, in a site where the slit 25 is not provided in the FZP member 20, the ultrasonic wave does not reach the skin of the living body.

In the ultrasonic wave passing through the slit 25 of the FZP member 20, ultrasonic wave interference occurs. Therefore, the FZP member 20 generates a diffraction phenomenon of the ultrasonic wave generated from the ultrasonic wave generation surface 10F and focuses the ultrasonic wave at a focal point which is the treatment site. That is, the FZP member 20 can focus energy of the ultrasonic wave on the focal point. Thus, the FZP member 20 can give a stimulus to the treatment site.

According to the ultrasonic wave unit 5 of the present embodiment, unlike the first embodiment described above, the gel member 30 is not disposed on the second FZP surface 20S, and thus the configuration of the gel member 30 can be simplified. Especially, it is not necessary to adjust the second thickness T2 of the gel member 30. By adjusting only the first thickness T1, the thickness of the gel member 30 can be adjusted. Therefore, it is easy to manage the thickness of the gel member 30.

### Fifth Embodiment

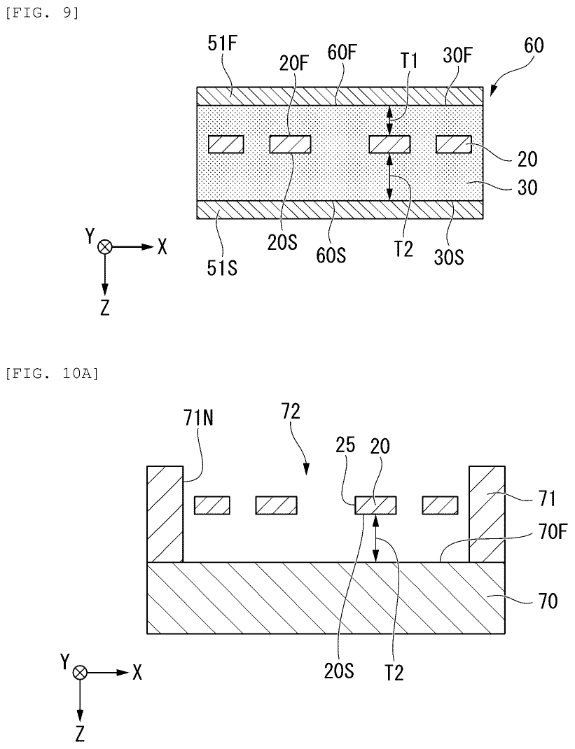

FIG. 7 is a schematic cross-sectional view partially showing a structure of an ultrasonic wave unit 6 according to a fifth embodiment, as viewed from a direction parallel to the ultrasonic wave unit 6.

The fifth embodiment is different from the first embodiment in the structure of the gel member 30.

As shown in FIG. 7, the ultrasonic wave unit 6 includes the piezoelectric element 10, the FZP member 20, and the gel member 30 as in the first embodiment. The gel member 30 includes a first gel member 30A and a second gel member 30B.

The first gel member 30A is an example of a first swelling body.

The second gel member 30B is an example of a second swelling body.

The first gel member 30A is positioned between the ultrasonic wave generation surface 10F and the second FZP surface 20S. The first gel member 30A is configured to adhere the FZP member 20 to the piezoelectric element 10. The second gel member 30B is attachable to and detachable from the second FZP surface 20S. In other words, the second gel member 30B is attachable to and detachable from the first gel member 30A. The first gel member 30A has the first thickness T1. The second gel member 30B has the second thickness T2.

The first gel member 30A and the second gel member 30B are made of the same material. In other words, a refractive index of the first gel member 30A and a refractive index of the second gel member 30B are preferably equal to each other.

Note that the first gel member 30A and the second gel member 30B may be made of different materials as long as a degree of inhibition of propagation of the ultrasonic wave can be sufficiently reduced.

The dotted line connecting the two points indicated by reference numeral A and reference numeral B in FIG. 7 indicates a surface between the first gel member 30A and the second gel member 30B. FIG. 7 shows that an interface is generated between the first gel member 30A and the second gel member 30B. This surface is a surface (peeling-off surface) where the second gel member 30B is peeled off from the first gel member 30A. On this surface, it is preferable that the first gel member 30A and the second gel member 30B are in close contact with each other, and an interface that inhibits propagation of the ultrasonic wave is not formed.

### <Functions and Effects>

According to the ultrasonic wave unit 6 of the present embodiment, after treatment using the ultrasonic wave unit 6 is completed, the used second gel member 30B in contact with the skin can be peeled off from the piezoelectric element 10 and discarded while the first gel member 30A disposed between the piezoelectric element 10 and the FZP member 20 remains. The ultrasonic wave unit 6 can be used by attaching a new second gel member 30B to the first gel member 30A. Since the second gel member 30B can be easily replaced, the ultrasonic wave unit 6 excellent in hygiene can be achieved.

### Sixth Embodiment

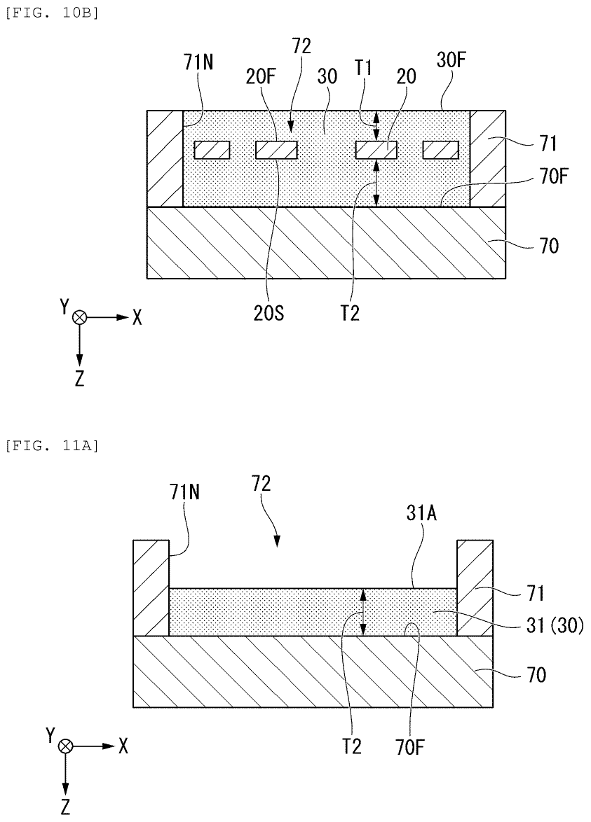

FIG. 8A is a schematic cross-sectional view partially showing a structure of a diffraction gel tape according to a sixth embodiment, as viewed from a direction parallel to a diffraction gel tape.

A diffraction gel tape 50 according to the present embodiment can be used as the FZP member 20 and the gel member 30 constituting each of the ultrasonic wave units 4 to 6 shown in FIGS. 5 to 7.

The diffraction gel tape 50 is an example of a diffraction swelling tape.

As shown in FIG. 8A, the diffraction gel tape 50 includes the FZP member 20 and the gel member 30. The gel member 30 covers at least one of the first FZP surface 20F and the second FZP surface 20S of the FZP member 20.

In the present embodiment, the gel member 30 covers the first FZP surface 20F and exposes the second FZP surface 20S. Note that the gel member 30 may cover the second FZP surface 20S. In this case, the gel member 30 exposes the second FZP surface 20S.

The diffraction gel tape 50 may be a tape extending in the Y direction. In this case, a plurality of FZP members 20 are arranged in the Y direction in the diffraction gel tape 50.

In the diffraction gel tape 50, a plurality of cuts for dividing the plurality of FZP members 20 one by one may be formed. In this case, the plurality of cuts are arranged in the Y direction.

Since the plurality of cuts are formed in advance in the diffraction gel tape 50 extending in the Y direction, the user who uses the diffraction gel tape 50 can easily separate the plurality of FZP members 20 one by one.

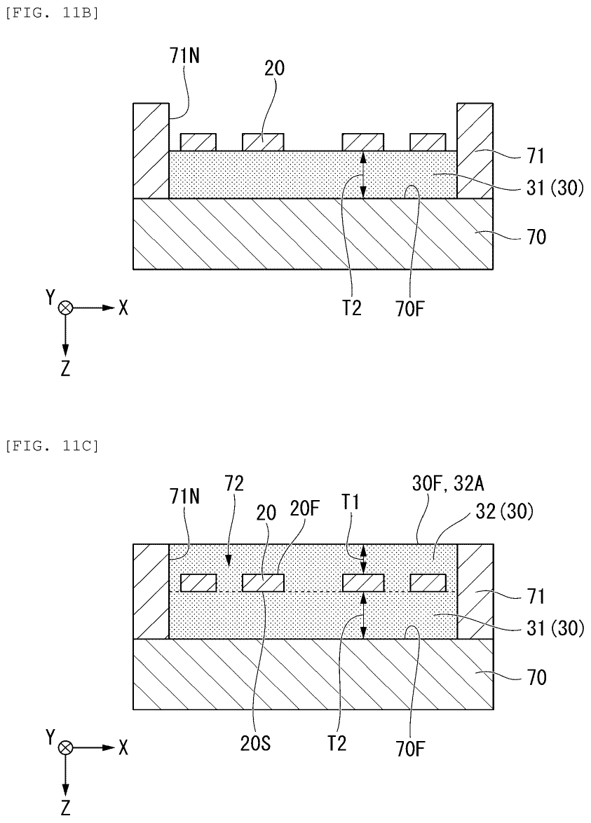

Note that in the following description, a cross section of a part of the diffraction gel tape extending in the Y direction will be described. The same applies to FIGS. 9 to 11 to be described later.

The diffraction gel tape 50 is mainly constituted by the gel member 30 and thus has adhesiveness. Therefore, the diffraction gel tape 50 can also be referred to as an adhesive tape. The diffraction gel tape 50 is a member in which the gel member 30 and the FZP member 20 are integrated, and thus may be referred to as a tape integrated body. Such a diffraction gel tape 50 can be directly and easily attached to the contact surface 30F of the piezoelectric element 10.

The diffraction gel tape 50 has a first tape surface 50F and a second tape surface 50S opposite to the first tape surface 50F. In the present embodiment, the first tape surface 50F is a surface corresponding to the contact surface 30F of the gel member 30. The second tape surface 50S is a surface corresponding to the exposed surface 30S of the gel member 30. Note that the first tape surface 50F may correspond to the exposed surface 30S. In this case, the second tape surface 50S corresponds to the contact surface 30F.

A distance between the first tape surface 50F and the first FZP surface 20F is a third thickness T3. The third thickness T3 corresponds to the first thickness T1 or the second thickness T2 described above. By adjusting the third thickness T3, the diffraction gel tape 50 can adjust the focal length or the convergence rate of the ultrasonic wave obtained by the ultrasonic wave unit.

In other words, a position of a surface of the diffraction gel tape 50 attached to the piezoelectric element 10 is not limited. In accordance with the configuration of the ultrasonic wave unit shown in FIGS. 5 to 7, the first tape surface 50F of the diffraction gel tape 50 may be attached to the piezoelectric element 10, or the second tape surface 50S of the diffraction gel tape 50 may be attached to the piezoelectric element 10. Note that in a case where the diffraction gel tape 50 is applied to the ultrasonic wave unit 6 shown in FIG. 7, the second tape surface 50S is a surface with which the application gel 40 comes into contact.

In the present embodiment, the diffraction gel tape 50 includes a first protective sheet 51F and a second protective sheet 51S. The first protective sheet 51F covers the first tape surface 50F. The second protective sheet 51S covers the second tape surface 50S.

Each of the first protective sheet 51F and the second protective sheet 51S has a function of protecting each of the FZP member 20 and the gel member 30. Each of the first protective sheet 51F and the second protective sheet 51S functions as a support that supports the FZP member 20, the gel member 30, and the diffraction gel tape 50.

### <Functions and Effects>

According to the diffraction gel tape 50 of the present embodiment, the same effects as those of the ultrasonic wave units 4 to 6 can be obtained. Furthermore, according to the diffraction gel tape 50, after treatment using the diffraction gel tape 50 is completed, the used diffraction gel tape 50 which is in contact with the skin can be peeled off from the piezoelectric element 10 and discarded. By attaching a new diffraction gel tape 50 to the piezoelectric element 10, the ultrasonic wave unit can be repeatedly used. Since the diffraction gel tape 50 can be easily replaced, the ultrasonic wave unit excellent in hygiene can be achieved.

Since both surfaces of the diffraction gel tape 50 are covered by the first protective sheet 51F and the second protective sheet 51S, adhesion of dust or dirt to the gel member 30 can be prevented before use of the ultrasonic wave unit.

Since the FZP member 20 and the gel member 30 are protected by the first protective sheet 51F and the second protective sheet 51S, the occurrence of damage to the FZP member 20 and the gel member 30 can be prevented.

Note that in the example shown in FIG. 8A, the first protective sheet 51F and the second protective sheet 51S are attached to both surfaces of the diffraction gel tape 50, but a configuration in which a protective sheet is attached to only one surface of the diffraction gel tape 50 may be adopted.

### <Modification of Diffraction Gel Tape 50>

FIG. 8B is a plan view partially showing a modification of the diffraction gel tape according to the sixth embodiment.

In the diffraction gel tape 50, a plurality of diffraction gel members 55 are disposed on the first protective sheet 51F. Each of the plurality of diffraction gel members 55 includes the FZP member 20 and the gel member 30 shown in FIG. 3B. The plurality of diffraction gel members 55 are arranged in an extending direction of the first protective sheet 51F, that is, along the Y direction. Each of the plurality of diffraction gel members 55 is disposed on the first protective sheet 51F in advance in an individualized state.

A cross-sectional structure of the diffraction gel member 55 is, for example, the structure shown in FIG. 8A.

Note that the plurality of diffraction gel members 55 may be connected by a connection portion 56 formed of the same material as the gel member 30.

When the ultrasonic wave unit is used, the user first peels off the diffraction gel member 55 from the first protective sheet 51F. Thereafter, the user attaches one surface of the diffraction gel member 55 to the skin of the living body. Further, the user attaches the piezoelectric element 10 to the other surface of the diffraction gel member 55. The signal cable 103 is connected to the piezoelectric element 10 in advance, for example. In this way, the ultrasonic wave unit can be implemented.

Note that the plurality of diffraction gel members 55 may be disposed on the second protective sheet 51S instead of the first protective sheet 51F.

After the piezoelectric element 10 is attached to one surface of the diffraction gel member 55, the other surface of the diffraction gel member 55 may be attached to the skin of the living body.

### Seventh Embodiment

FIG. 9 is a schematic cross-sectional view partially showing a structure of a diffraction gel tape according to a seventh embodiment, as viewed from a direction parallel to the diffraction gel tape.

In the seventh embodiment, the same members as those of the sixth embodiment are denoted by the same reference numerals, and the description thereof is omitted or simplified.

A diffraction gel tape 60 according to the present embodiment can be used as the FZP member 20 and the gel member 30 constituting the ultrasonic wave unit 1A shown in FIG. 2 and FIG. 3B.

The diffraction gel tape 60 is an example of a diffraction swelling tape.

As shown in FIG. 9, the diffraction gel tape 60 includes the FZP member 20 and the gel member 30 described in the first embodiment. The diffraction gel tape 60 is different from the above-described diffraction gel tape 50 in that the gel member 30 covers both the first FZP surface 20F and the second FZP surface 20S of the FZP member 20. The description of the same configuration as that of the diffraction gel tape 50 is omitted.

The diffraction gel tape 60 has a first tape surface 60F and a second tape surface 60S opposite to the first tape surface 60F. In the present embodiment, the first tape surface 60F is a surface corresponding to the contact surface 30F of the gel member 30. That is, the first tape surface 60F is a surface that comes into contact with the ultrasonic wave generation surface 10F. The second tape surface 60S is a surface corresponding to the exposed surface 30S of the gel member 30.

The first protective sheet 51F covers the first tape surface 60F. The second protective sheet 51S covers the second tape surface 60S.

A distance between the first tape surface 60F and the first FZP surface 20F is the first thickness T1. A distance between the second tape surface 60S and the second FZP surface 20S is the second thickness T2. By adjusting one of the first thickness T1 and the second thickness T2, the diffraction gel tape 60 can adjust the focal length or the convergence rate of the ultrasonic wave obtained by the ultrasonic wave unit 1.

### <Modification of Diffraction Gel Tape 60>

The diffraction gel tape 60 may be applied to the modification shown in FIG. 8B described above.

### <Method for Manufacturing Diffraction Gel Tape 60>

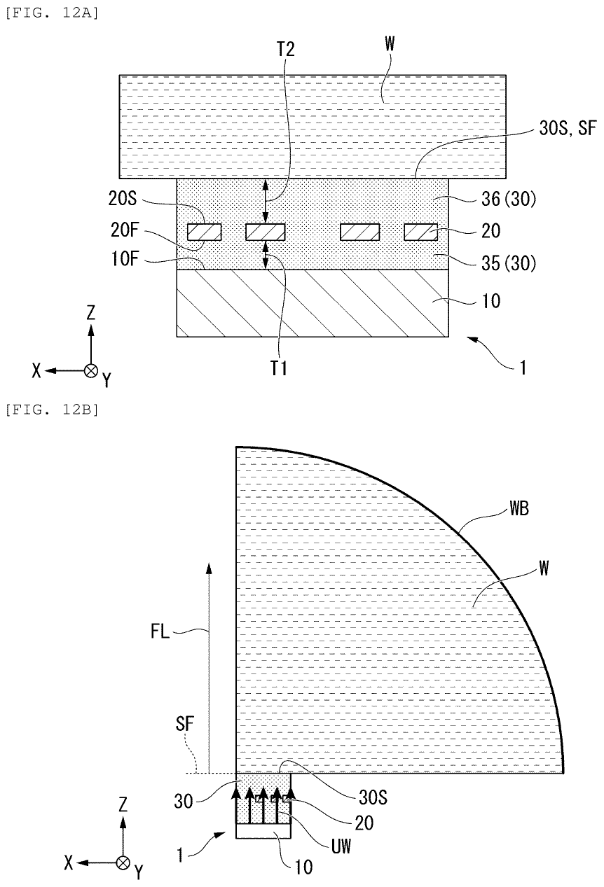

FIGS. 10A and 10B are schematic cross-sectional view partially showing the structure of the diffraction gel tape 60 according to the seventh embodiment, and are views illustrating a method for manufacturing the diffraction gel tape 60.

First, as shown in FIG. 10A, a base 70 and a frame 71 are prepared. The base 70 has a base surface 70F. The frame 71 has a frame inner surface 71N. A region surrounded by the base surface 70F and the frame inner surface 71N is a mold 72 for molding the diffraction gel tape 60.

The base 70 and the frame 71 are made of a material excellent in peeling property and liquid repellency to the urethane resin constituting the gel member 30. As such a material, a material such as a resin material or a metal material is used. Note that the base surface 70F and the frame inner surface 71N may have peeling property and liquid repellency to the constituent material of the gel member 30.

In a state in which the frame 71 is disposed on the base surface 70F of the base 70, the FZP member 20 is disposed inside the mold 72. A height from the base surface 70F to the second FZP surface 20S of the FZP member 20 corresponds to the above-described second thickness T2. This height is set by a jig or the like that is used repeatedly only in the method for manufacturing the diffraction gel tape. A spacer formed of the material constituting the FZP member 20 may be disposed between the base surface 70F and the FZP member 20.

In this case, the spacers may be arranged in the Y direction in which the diffraction gel tape 60 extends. For example, the spacers may be formed near the positions of the plurality of cuts formed in the diffraction gel tape 50. The spacer may be separable from the FZP member 20. For example, when the plurality of FZP members 20 formed on the diffraction gel tape 60 are easily separated one by one, the spacer may have a shape or configuration separable from the FZP member 20.

Next, as shown in FIG. 10B, the urethane resin as the material of the gel member 30 is poured into the mold 72. In this way, the urethane resin material covers the entire FZP member 20 and fills the inside of the slit 25. A distance between a liquid surface serving as the contact surface 30F of the gel member 30 and the first FZP surface 20F corresponds to the above-described first thickness T1. Thereafter, the urethane resin material is cured. A method for curing the urethane resin material is not particularly limited. A curing method in accordance with the type of the urethane resin material is used. Thereafter, the base 70 and the frame 71 are removed from the cured urethane resin material member. Thereafter, the first protective sheet 51F is attached to the first tape surface 60F. Further, the second protective sheet 51S is attached to the second tape surface 60S. Thus, the diffraction gel tape 60 shown in FIG. 9 is obtained.

### <Functions and Effects>

According to the diffraction gel tape 60 of the present embodiment, the same effects as those of the ultrasonic wave unit 1 can be obtained. Furthermore, according to the diffraction gel tape 60, after treatment using the diffraction gel tape 60 is completed, the used diffraction gel tape 60 which is in contact with the skin can be peeled off from the piezoelectric element 10 and discarded. By attaching a new diffraction gel tape 60 to the piezoelectric element 10, the ultrasonic wave unit 1 can be repeatedly used. Since the diffraction gel tape 60 can be easily replaced, the ultrasonic wave unit 1 excellent in hygiene can be achieved.

Note that in the method for manufacturing the above-described diffraction gel tape 60, a chamber capable of adjusting an internal pressure may be used to prevent air bubbles from mixing into the interior of the diffraction gel tape 60. In this case, by manufacturing the diffraction gel tape 60 in a reduced-pressure atmosphere in the chamber, it is possible to manufacture the diffraction gel tape 60 in which mixing of air bubbles is prevented. Accordingly, it is possible to reduce the attenuation of the ultrasonic wave caused by the mixing of the air bubbles.

### <Modification of Method for Manufacturing Diffraction Gel Tape 60>

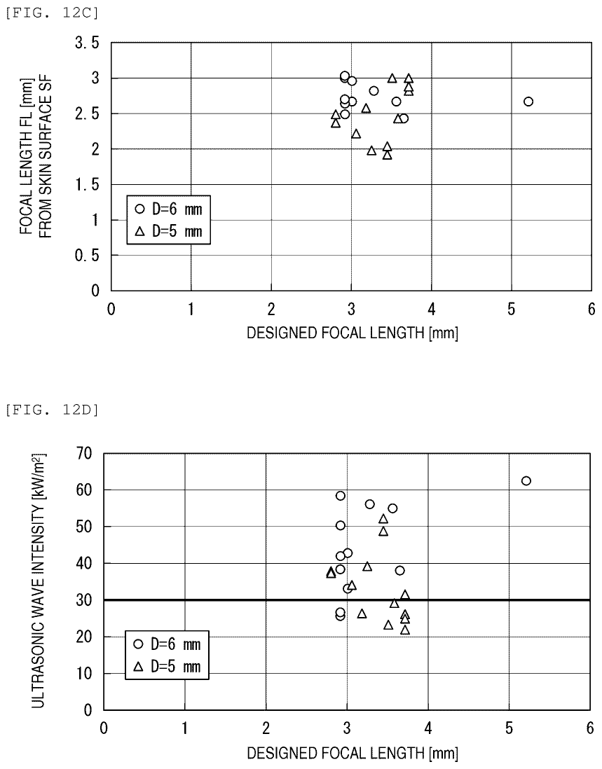

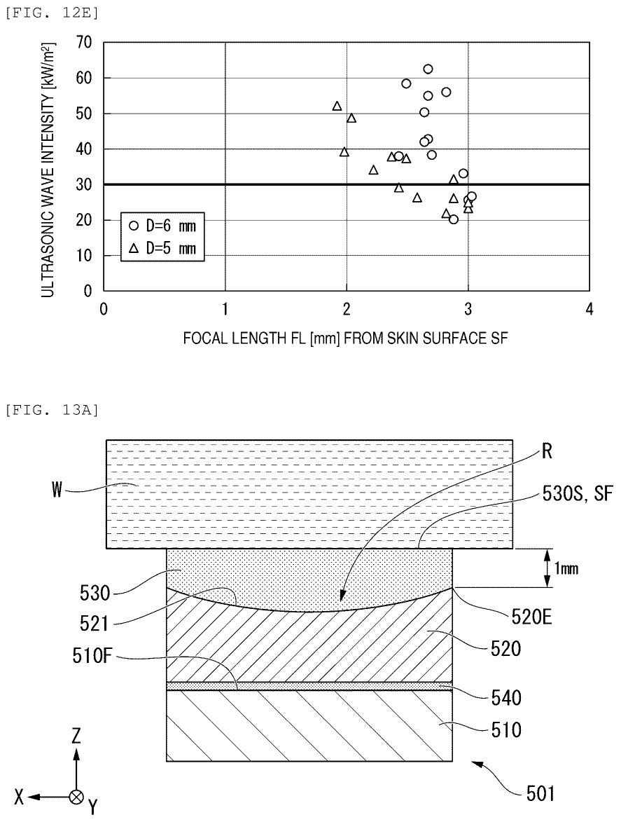

Next, a modification of the method for manufacturing the diffraction gel tape 60 will be described with reference to FIGS. 11A to 11C.

In the present modification, the same members as those in FIGS. 10A and 10B are denoted by the same reference numerals, and description thereof is omitted or simplified.

First, as shown in FIG. 11A, in a state in which the frame 71 is disposed on the base surface 70F of the base 70, inside the mold 72, a first urethane resin 31 which is the material of the gel member 30 is poured into the mold 72. A position of an upper surface 31A of the first urethane resin 31 in the Z direction is set so as to obtain the second thickness T2 of the gel member 30. In other words, a distance between the upper surface 31A and the base surface 70F corresponds to the second thickness T2. Thereafter, the first urethane resin 31 is cured.

Next, the FZP member 20 is disposed on the upper surface 31A of the first urethane resin 31. In this way, a height from the base surface 70F to the FZP member 20 in the Z direction is determined. In the present modification, the spacer for setting the height of the FZP member 20 can be omitted. A method for disposing the FZP member 20 on the upper surface 31A is not limited.

Next, as shown in FIG. 11C, a second urethane resin 32, which is the material of the gel member 30, is poured into the mold 72. In this way, the second urethane resin 32 covers the entire FZP member 20 and fills the inside of the slit 25. A distance between a liquid surface 32A of the second urethane resin 32 serving as the contact surface 30F of the gel member 30 and the first FZP surface 20F corresponds to the above-described first thickness T1. Thereafter, the second urethane resin 32 is cured. Thereafter, the base 70 and the frame 71 are removed from the first urethane resin 31 and the second urethane resin 32 after curing. Thus, the diffraction gel tape 60 shown in FIG. 9 is obtained.

According to the present modification, the position of the FZP member 20 in the Z direction can be easily set.

### <Modification of Ultrasonic Wave Unit>

In the above-described embodiments, a configuration in which the ultrasonic wave focusing device 200 drives one ultrasonic wave unit is described. The ultrasonic wave units of the above-described embodiments may be applied to an ultrasonic wave unit array in which a plurality of ultrasonic wave units are arranged.

Examples of the ultrasonic wave unit array include a one-dimensional ultrasonic wave unit array in which a plurality of ultrasonic wave units are arranged one-dimensionally. Specifically, a configuration in which a plurality of ultrasonic wave units are arranged in the X direction or the Y direction can be adopted.

Examples of the ultrasonic wave unit array include a two-dimensional ultrasonic wave unit array in which a plurality of ultrasonic wave units are arranged two-dimensionally. Specifically, it is possible to adopt a configuration in which a plurality of ultrasonic wave units are arranged in both the X direction and the Y direction. For example, in a structure in which a two-dimensional ultrasonic wave unit array is attached to a planar sheet, a planar array can be implemented. In a structure in which a two-dimensional ultrasonic wave unit array is attached to a deformable or stretchable sheet, a curved surface array can be implemented.

As described above, in the configuration in which the diffraction gel tape is attached to the piezoelectric element, the plurality of ultrasonic wave units can be easily applied to the two-dimensional ultrasonic wave unit array.

### <Application Example of Ultrasonic Wave Unit, Diffraction Gel Tape, and Ultrasonic Wave Focusing Device>

The above embodiments describe the cases where the ultrasonic wave unit, the diffraction gel tape, and the ultrasonic wave focusing device are applied to the acupuncture point stimulation device.

The invention is not limited to the acupuncture point stimulation device that stimulates the treatment site, and may be used for treatment, diagnosis, surgery, and the like with respect to a living body.

For example, by using a measurement unit in which the above-described ultrasonic wave unit and a reflected wave sensor for detecting a reflected wave of ultrasonic wave are combined, it is possible to implement various types of diagnosis devices and measurement devices relating to medical care. Hereinafter, examples in which the invention is applied will be listed.

### (1) Ultrasonic Wave Sensor

An ultrasonic wave sensor includes a measurement unit provided with an ultrasonic wave unit and a reflected wave sensor. The ultrasonic wave unit may be configured to have not only the above-described function of focusing the ultrasonic wave but also a function of a reflected wave sensor.

The ultrasonic wave sensor can be used as a sensor capable of changing a measurement position in a body. For example, the sensor can be attached to a body surface directly above a blood vessel of a living body, and various kinds of information related to behaviors of the blood vessel and movement of blood can be obtained.

### (2) Sheet-Like Ultrasonic Wave Probe

A sheet-like ultrasonic wave probe includes a plurality of measurement units each provided with an ultrasonic wave unit and a reflected wave sensor. For example, the sheet-like ultrasonic wave probe may be a sheet in which a plurality of ultrasonic wave units configured to have not only the above-described function of focusing the ultrasonic wave but also a function of a reflected wave sensor are arranged. The array of the plurality of ultrasonic wave units may be a one-dimensional array arranged in one line, or a two-dimensional array arranged in the X direction and the Y direction. Such an ultrasonic wave probe is attached to a body surface of a living body. The sheet-like ultrasonic wave probe may be deformable according to the shape of the body surface. The sheet-like ultrasonic wave probe may have flexibility capable of being bent in a predetermined direction for beam forming of ultrasonic waves, collimation of ultrasonic waves, and modulation of ultrasonic waves. Such an ultrasonic wave probe can measure carotid artery and jugular vein of a living body by, for example, being attached to the neck. Further, by attaching the ultrasonic wave probe to the body surface on the clavicle, it is possible to obtain various kinds of information concerning the blood in the inferior vena cava and the inferior aorta.

Furthermore, such an ultrasonic wave probe can be used for measuring the shape and movement of heart by passing through the ribs and being disposed in the living body. The ultrasonic wave probe can obtain various kinds of information relating to heart. Further, it is also possible to perform image processing such as imaging of heart based on information obtained by the ultrasonic wave probe.

By attaching the ultrasonic wave probe to a surface of neck, the measurement of movement of throat and evaluation of muscle for moving throat lid can be performed.

### EXAMPLES

Next, effects of the invention will be specifically described with reference to Examples.

In the following, a case in which the ultrasonic wave unit is applied to an acupuncture point stimulation device will be described. In this case, differences between an ultrasonic wave unit in the related art including an acoustic lens having a concave surface and the ultrasonic wave unit of the invention will be described.

### Example 1

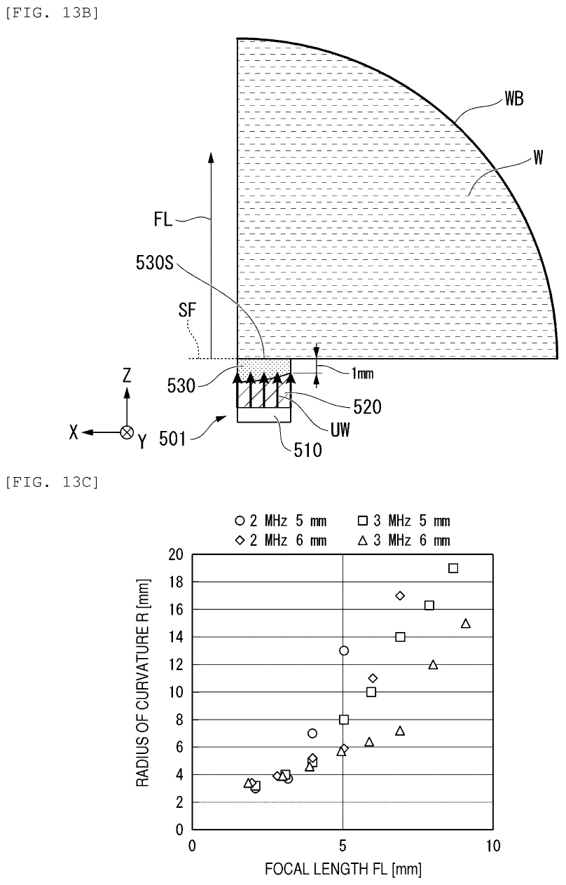

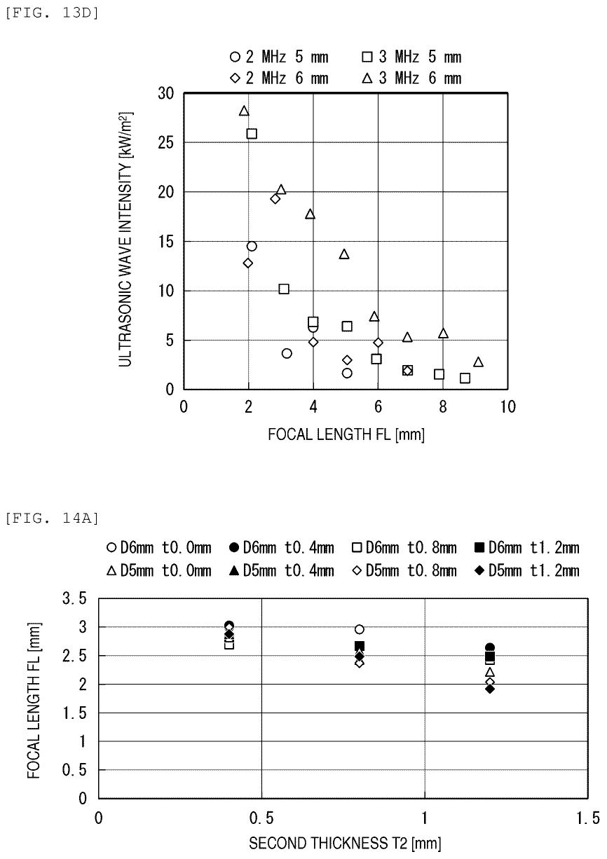

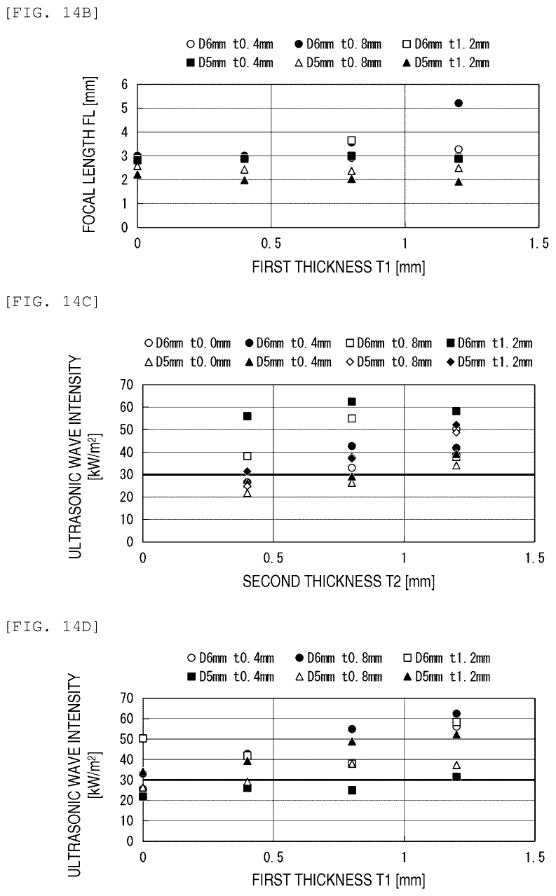

FIG. 12A is a cross-sectional view schematically showing an ultrasonic wave unit according to Examples 1 and 2, and is a view illustrating conditions of simulation.

FIG. 12B is a cross-sectional view schematically showing the ultrasonic wave unit according to Example 1, and is a view illustrating an analysis model for the simulation.

FIG. 12C is a diagram showing results of the simulation of the ultrasonic wave unit according to Example 1, which is a graph showing a relation between a designed focal length (mm) and a focal length FL (mm) from a skin surface SF.

FIG. 12D is a diagram showing results of the simulation of the ultrasonic wave unit according to Example 1, which is a graph showing a relation between the designed focal length (mm) and an ultrasonic wave intensity (kW/m²).

FIG. 12E is a diagram showing results of the simulation of the ultrasonic wave unit according to Example 1, which is a graph showing a relation between the focal length FL (mm) from the skin surface SF and the ultrasonic wave intensity (kW/m²).

In the simulation of Example 1, the ultrasonic wave unit 1 shown in FIG. 3A is used.

The reference numeral W indicates water used as an example of the skin in the simulation.

The reference numeral WB indicates an open interface.

The reference numeral SF coincides with the exposed surface 30S of the gel member 30 constituting the ultrasonic wave unit 1, and indicates the skin surface. On the skin surface SF, the exposed surface 30S is in contact with the water W.

The reference numeral FL indicates the focal length. That is, the focal length FL means a distance from the skin surface SF toward the inside of the skin.

The reference numeral UW indicates an ultrasonic wave generated from the ultrasonic wave generation surface 10F.

In the simulation of Example 1, the frequency of the ultrasonic wave generated from the piezoelectric element 10 was set to 3 MHz. A sound pressure was set to 2.8×10⁴ Pa. Here, the sound pressure means a pressure generated on the ultrasonic wave generation surface of the piezoelectric element 10. The FZP member 20 was formed using an acrylic resin. The gel member 30 was formed using a human skin gel stock solution (registered trademark). A density of the gel member 30 is approximately 998.6 kg/m³, and a propagation speed of the ultrasonic wave in the gel member 30 is 1141.5 m/s.

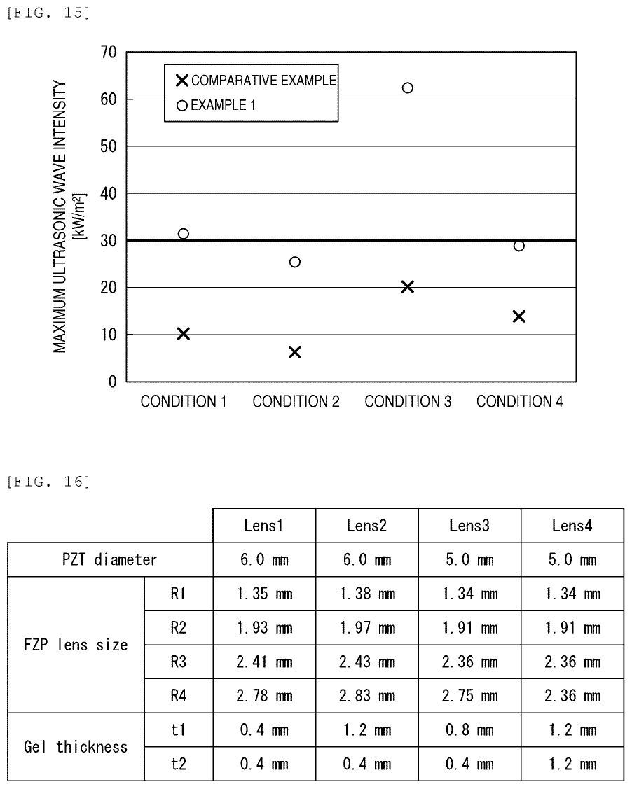

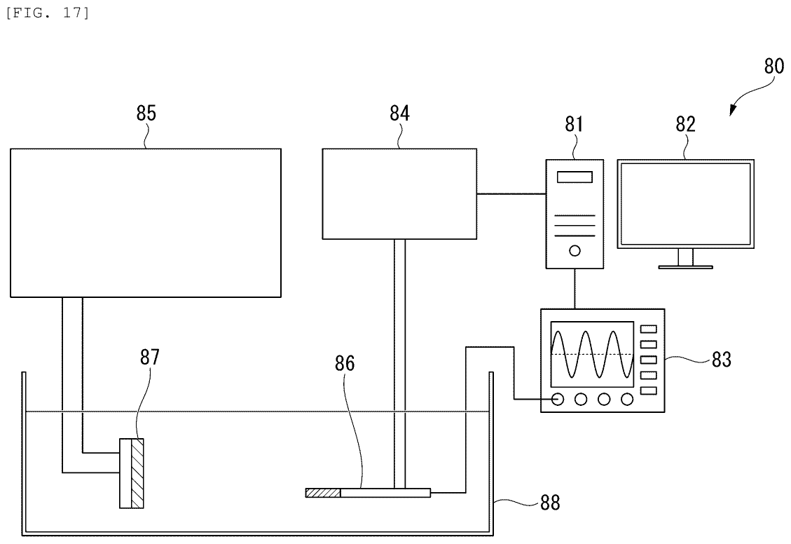

In each of FIGS. 12C, 12D, and 12E, 13 samples in which a diameter of the piezoelectric element 10 was 5 mm were prepared, and 13 samples in which a diameter of the piezoelectric element 10 was 6 mm were prepared, and simulation was performed. In this way, the intensity of the ultrasonic wave propagated from the surface of the skin, which is a body surface, toward the inside of the skin was analyzed.

Regarding the designed focal length, the designed focal length was adjusted so that the focal length FL was approximately 3 mm and the strongest ultrasonic wave intensity was obtained. Here, in the adjustment of the designed focal length, the thickness of the FZP member 20, the width of the slit 25, and the thickness of the gel member 30 were adjusted.

Since the frequency of the ultrasonic wave was 3 MHZ, the thickness of the FZP member 20 was set to 0.4 mm. The width of the slit 25 was adjusted within a range of 0.4 mm to 1.0 mm. The thickness of the gel member 30 was adjusted within a range of 0.4 mm to 2.4 mm.

In FIGS. 12D and 12E, a position where the ultrasonic wave intensity is 30 kW/m² is indicated by a bold line. This intensity refers to an ultrasonic wave intensity that was found to be actually effective when a test was conducted in which acupuncture point stimulation was performed using a focused ultrasonic wave by an acupuncture point stimulation device equipped with an ultrasonic wave unit. Specifically, it means that the ultrasonic wave intensity is 30 kW/m² at the position of the focal point.

Note that the meaning of the ultrasonic wave intensity of 30 kW/m² indicated by a bold line in the views illustrating Comparative Example, Example 2, and Example 3 to be described later is the same as that in FIGS. 12D and 12E.

### (Evaluation Results)

From the results shown in FIGS. 12C, 12D, and 12E, the following points were revealed.

(A1) It is clear that the focal length FL from the skin surface SF can be adjusted by adjusting the designed focal length.

(A2) It is clear that in Example 1, the designed focal length was adjusted so that the focal length FL was approximately 3 mm and the strongest ultrasonic wave intensity was obtained, but it was possible to set the focal length FL to 3 mm or more by adjusting the designed focal length.

(A3) It is clear that by adjusting the designed focal length, that is, when the focal length FL is approximately 3 mm, the ultrasonic wave intensity exceeding 30 kW/m² at which the effect of acupuncture point stimulation is recognized can be obtained.

### Comparative Example

FIG. 13A is a cross-sectional view schematically showing an ultrasonic wave unit of Comparative Example, and is a diagram illustrating conditions of simulation.

FIG. 13B is a cross-sectional view schematically showing the ultrasonic wave unit of Comparative Example, and is a view illustrating an analysis model for the simulation.

FIG. 13C is a diagram showing results of the simulation of the ultrasonic wave unit of Comparative Example, which is a graph showing a relation between the focal length FL (mm) and a radius of curvature (mm) of the acoustic lens.

FIG. 13D is a diagram showing results of the simulation of the ultrasonic wave unit of Comparative Example, which is a graph showing a relation between the focal length FL (mm) and the ultrasonic wave intensity (kW/m²).

In the simulation of Comparative Example, an ultrasonic wave unit 501 including an acoustic lens having a concave surface is used.

The ultrasonic wave unit 501 includes a piezoelectric element 510 having an ultrasonic wave generation surface 510F, an acoustic lens 520 having a concave surface 521, a gel member 530 provided on a surface of the concave surface 521, and an attachment tape 540 for adhering the acoustic lens 520 to the ultrasonic wave generation surface 510F.

The gel member 530 has an exposed surface 530S in contact with the water W used as an example of the skin in the simulation. The exposed surface 530S coincides with the skin surface SF.

The piezoelectric element 510 has the same configuration as the piezoelectric element 10 constituting the ultrasonic wave unit 1.

The focal length FL is a distance from the skin surface SF toward the inside of the skin. The ultrasonic wave UW is generated from the ultrasonic wave generation surface 510F. A distance between an edge 520E of the acoustic lens 520 and the skin surface SF is set to 1 mm. The acoustic lens 520 has a radius of curvature R (mm). In Comparative Example, the radius of curvature R was adjusted within a range of 3 to 19.

In the simulation of Comparative Example, a frequency of the ultrasonic wave generated from the piezoelectric element 510 was set to 2 MHz or 3 MHZ. The sound pressure was set to 1.0×10⁵ Pa. Here, the sound pressure means a pressure generated on the ultrasonic wave generation surface of the piezoelectric element 510. The acoustic lens 520 was formed using an acrylic resin. A material of the gel member 530 is the same as that of the gel member 30.

In each of FIGS. 13C and 13D, a total of 26 samples were prepared and simulation was performed. In this way, an intensity of the ultrasonic wave propagated from the surface of the skin, which is a body surface, toward the inside of the skin was analyzed. Specifically, four samples in which the frequency of the ultrasonic wave was set to 2 MHz and the diameter of the piezoelectric element 10 was 5 mm were prepared (“∘ 2 MHZ 5 mm”). Eight samples in which the frequency of the ultrasonic wave was set to 3 MHz and the diameter of the piezoelectric element 10 was 5 mm were prepared (“□ 3 MHz 5 mm”). Six samples in which the frequency of the ultrasonic wave was set to 2 MHz and the diameter of the piezoelectric element 10 was 6 mm were prepared (“⋄ 2 MHz 6 mm”). Eight samples in which the frequency of the ultrasonic wave was set to 3 MHz and the diameter of the piezoelectric element 10 was 6 mm were prepared (“Δ 3 MHZ 6 mm”).

### (Evaluation Results)

From the results shown in FIGS. 13C and 13D, the following points are clear.

(B1) In Comparative Example, the focal length FL is adjusted by adjusting the radius of curvature R of the acoustic lens 520. It is clear that the focal length FL becomes small by decreasing the radius of curvature R, and the focal length FL becomes large by increasing the radius of curvature R.

(B2) It is clear that the ultrasonic wave intensity decreases by increasing the focal length FL.

(B3) It is clear that when the frequency was small, the ultrasonic wave intensity was low.

(B4) It is clear that although the ultrasonic wave intensity was increased by decreasing the focal length FL, the ultrasonic wave intensity exceeding 30 kW/m² cannot be obtained. That is, it was found that the effect of the acupuncture point stimulation cannot be obtained.

### Example 2

As shown in FIG. 12A, the gel member 30 constituting the ultrasonic wave unit 1 according to Example 2 has a first gel region 35 having the first thickness T1 and a second gel region 36 having the second thickness T2. The first gel region 35 is positioned between the first FZP surface 20F and the ultrasonic wave generation surface 10F. The second gel region 36 is positioned between the second FZP surface 20S and the skin surface SF (exposed surface 30S).

In Example 2, the focal length or the ultrasonic wave intensity in each of the first thickness T1 and the second thickness T2 was analyzed. The conditions of the simulation of Example 2 are the same as those of Example 1 described above. A simulation was performed on a case where the diameter of the piezoelectric element 10 was 5 mm and a case where the diameter of the piezoelectric element 10 was 6 mm.

FIG. 14A is a diagram showing results of the simulation of the ultrasonic wave unit according to Example 2, which is a graph showing a relation between the focal length FL (mm) and the second thickness T2 (mm) of the second gel region 36.

FIG. 14B is a diagram showing results of the simulation of the ultrasonic wave unit according to Example 2, which is a graph showing a relation between the focal length FL (mm) and the first thickness T1 (mm) of the first gel region 35.

FIG. 14C is a diagram showing results of the simulation of the ultrasonic wave unit according to Example 2, which is a graph showing a relation between the second thickness T2 (mm) of the second gel region 36 and the ultrasonic wave intensity (kW/m²).

FIG. 14D is a diagram showing results of the simulation of the ultrasonic wave unit according to Example 2, which is a graph showing a relation between the first thickness T1 (mm) of the first gel region 35 and the ultrasonic wave intensity (kW/m²).

In each of FIGS. 14A, 14B, 14C, and 14D, the symbol “D5 mm” indicates case where the diameter of the piezoelectric element 10 is 5 mm. The symbol “D6 mm” indicates a case where the diameter of the piezoelectric element 10 is 6 mm. The symbol “t0.0 mm” indicates a case where the thickness of the gel region is 0.0 mm. The symbol “t0.4 mm” indicates a case where the thickness of the gel region is 0.4 mm. The symbol “t0.8 mm” indicates a case where the thickness of the gel region is 0.8 mm. The symbol “t1.2 mm” indicates a case where the thickness of the gel region is 1.2 mm.

### (Evaluation Results)

From the results shown in FIGS. 14A, 14B, 14C, and 14D, the following points are clear.

(C1) It is clear that the focal length FL can be adjusted by adjusting the second thickness T2. Especially, it is clear that the focal length FL can be reduced by increasing the second thickness T2.

(C2) It is clear that the change in the focal length FL is observed after the adjustment of the first thickness T1, but the adjustment of the first thickness T1 does not significantly affect the focal length FL.

(C3) It is clear that the focal length FL can be adjusted not only by simply adjusting the thickness of the gel member 30 including the FZP member 20 but also by adjusting the thicknesses of the first gel region 35 and the second gel region 36.

(C4) It is clear that the ultrasonic wave intensity can be adjusted by adjusting the second thickness T2. In other words, it is clear that the convergence rate of the ultrasonic wave can be adjusted. Especially, it is clear that the ultrasonic wave intensity can be increased by increasing the second thickness T2.

(C5) It is clear that the ultrasonic wave intensity can be adjusted by adjusting the first thickness T1. In other words, it is clear that the convergence rate of the ultrasonic waves can be adjusted. Especially, it is clear that the ultrasonic wave intensity can be increased by increasing the first thickness T1.

(C6) In order to obtain the ultrasonic wave intensity exceeding 30 kW/m² at which the effect of acupuncture point stimulation is recognized, it is clear that it is effective to adjust the thicknesses of the first gel region 35 and the second gel region 36.

### Example 3

FIG. 15 is a diagram showing results of comparison between Example 1 and Comparative Example on a maximum ultrasonic wave intensity under Conditions 1 to 4. In Condition 1, the diameter of the piezoelectric element 10 is 5 mm, and the focal length FL is 3 mm. In Condition 2, the diameter of the piezoelectric element 10 is 5 mm, and the focal length FL is 5 mm. In Condition 3, the diameter of the piezoelectric element 10 is 6 mm, and the focal length FL is 3 mm. In Condition 4, the diameter of the piezoelectric element 10 is 6 mm, and the focal length FL is 5 mm.

### (Evaluation Results)

From the results shown in FIG. 15, the following points are clear.

(D1) Under each of Conditions 1 to 4, it is clear that Example 1 provides a maximum ultrasonic wave intensity higher than that of Comparative Example. Especially, under Conditions 1, 3, and 4, it is clear that Example 1 provides a high ultrasonic wave intensity of 30 kW/m² or more at which the effect of acupuncture point stimulation is recognized.

(D2) In Comparative Example, it is clear that the ultrasonic wave intensity exceeding 30 kW/m² cannot be obtained.

### Example 4

Example 4 will be described with reference to FIGS. 16 to 30.

FIG. 17 is a diagram showing a configuration of a test device used in Example 4.

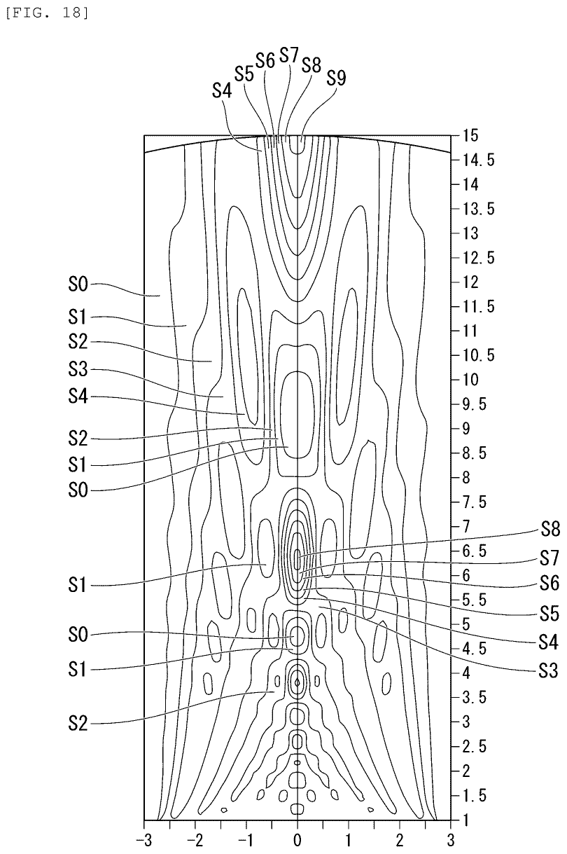

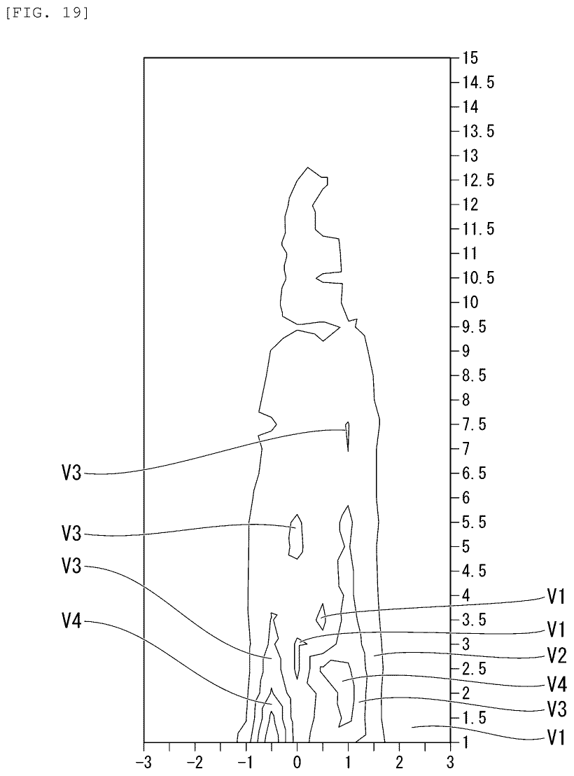

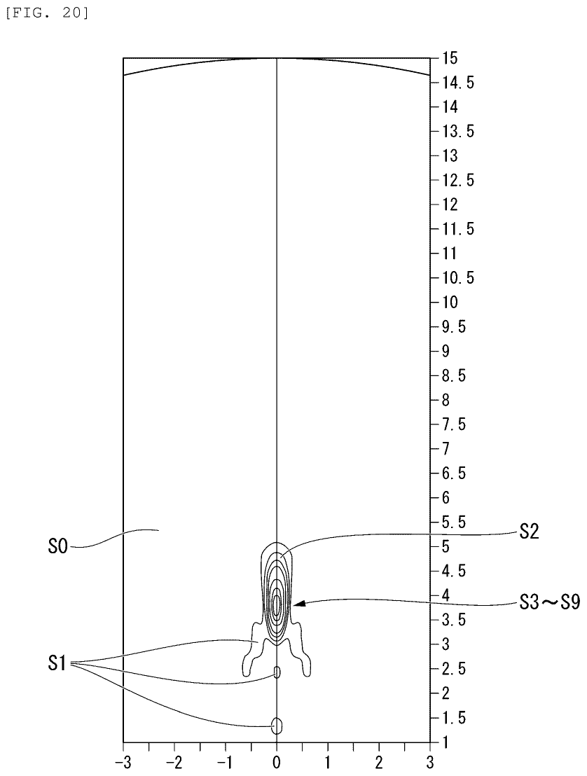

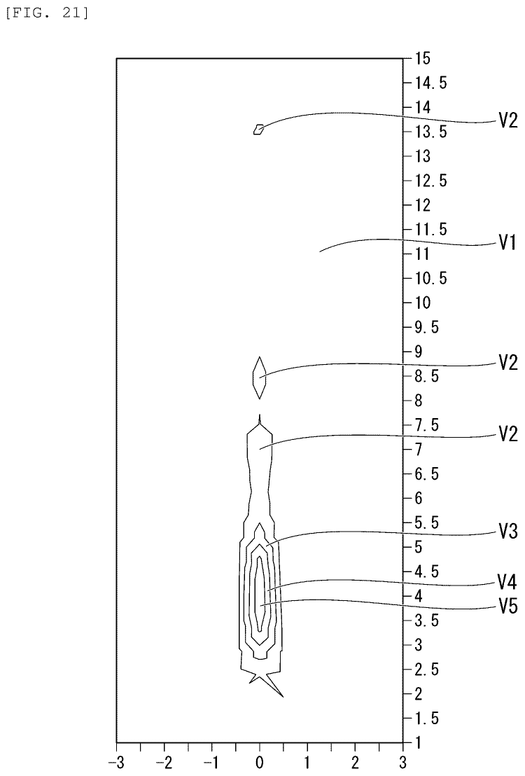

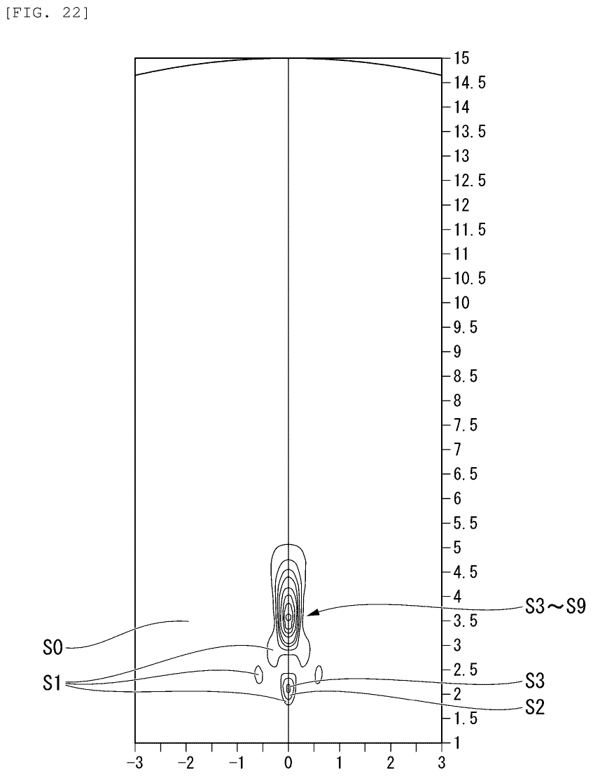

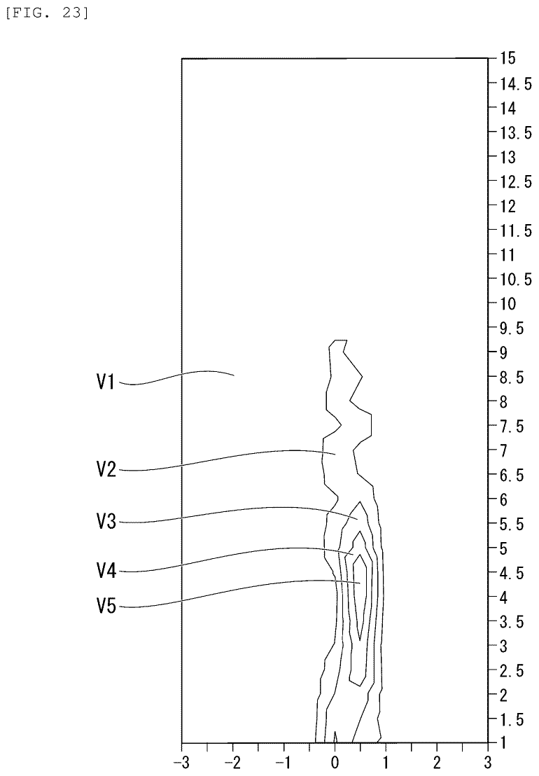

FIGS. 18 to 30 show simulation results and experimental results using a test device 80. In the following description, the experimental results using the test device 80 may be simply referred to as “experimental results”.

Based on the simulation results obtained according to Examples 1 to 3 described above, the effects of the invention were further verified in Example 4. Here, a simulation was performed on examples relating to four lenses and Comparative Example not using a lens (FZP member). Further, the FZP member and the gel member constituting the ultrasonic wave unit were actually manufactured. Each of the manufactured four lenses is an integrated body of the FZP member and the gel member described in the above-described embodiment. In the following description, the four lenses are referred to as lenses 1 to 4.

FIG. 16 is a table showing conditions of the lenses 1 to 4 (lens 1 to lens 4).

The “PZT diameter” means the diameter of the piezoelectric element 10.

R1 to R4 described in “FZP lens size” correspond to R1 to R4 of the FZP member 20 shown in FIG. 3B.

The reference numeral R1 indicates a distance from a center O of the ultrasonic wave unit 1A to an inner edge of the inner FZP portion 21 in the radial direction, that is, an inner diameter of the inner FZP portion 21.

The reference numeral R2 indicates a distance from the center O of the ultrasonic wave unit 1A to an outer edge of the inner FZP portion 21 in the radial direction, that is, an outer diameter of the inner FZP portion 21.

The reference numeral R3 indicates a distance from the center O of the ultrasonic wave unit 1A to an inner edge of the outer FZP portion 22 in the radial direction, that is, an inner diameter of the outer FZP portion 22.

The reference numeral R4 indicates a distance from the center O of the ultrasonic wave unit 1A to an outer edge of the outer FZP portion 22 in the radial direction, that is, an outer diameter of the outer FZP portion 22. In other words, the reference numeral R4 is a distance from the center O of the ultrasonic wave unit 1A to an outer peripheral edge of the FZP member 20 in the radial direction, that is, the diameter of the FZP member 20.

The “Gel thickness” means the thickness of the gel member 30 shown in FIG. 12A. The “t1” corresponds to the first thickness T1 and is the thickness of the first gel region 35. The “t2” corresponds to the second thickness T2 and is the thickness of the second gel region 36.

The lenses 1 to 4 are different from each other in terms of “PZT diameter”, “FZP lens s size”, and “Gel thickness”.

The lenses 1 to 4 used in the experiment using the test device 80 are different from those used in the simulation in that the coupling portion 23 is used. Specifically, in the lenses 1 to 4 used in the experiment using the test device 80, the inner FZP portion 21 and the outer FZP portion 22 are connected to each other by the coupling portion 23 as shown in FIG. 3B. On the other hand, in each of the lenses 1 to 4 used in the simulation, a structure in which the inner FZP portion 21 and the outer FZP portion 22 are not connected to each other by the coupling portion 23 is applied.

### <Simulation Results>

Each of FIGS. 18, 20, 22, 24, 26, and 28 shows the following simulation results.

FIG. 18: simulation results of Comparative Example using only a piezoelectric element having a diameter of 6 mm without using a lens (FZP member)

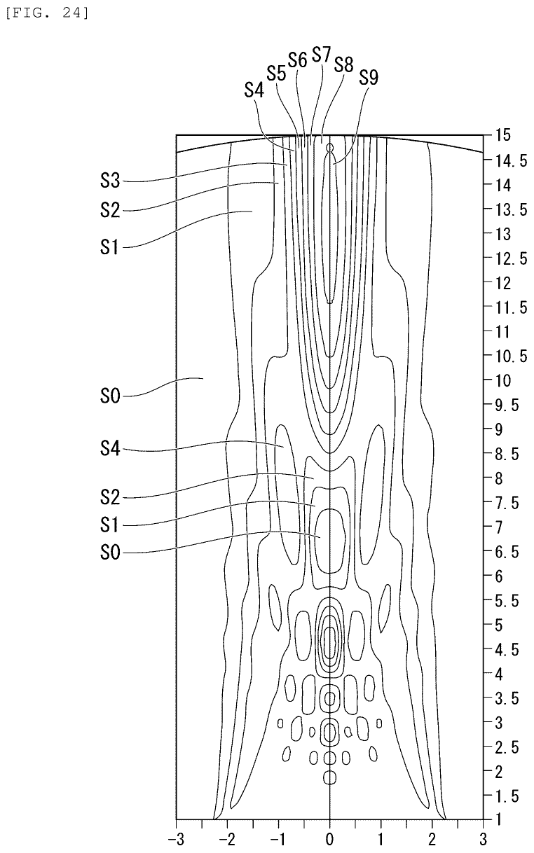

FIG. 20: simulation results of the lens 1

FIG. 22: simulation results of the lens 2

FIG. 24: simulation results of Comparative Example using only a piezoelectric element having a diameter of 5 mm without using a lens (FZP member).

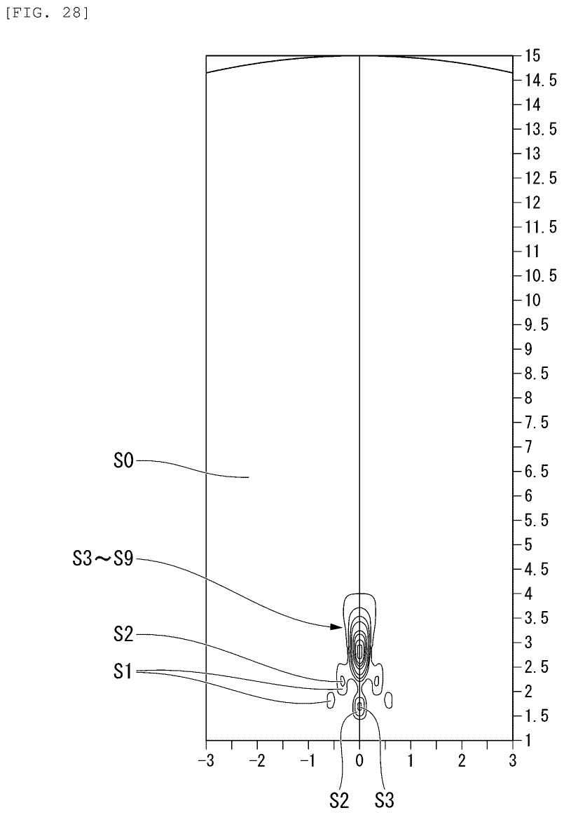

FIG. 26: simulation results of the lens 3

FIG. 28: simulation results of the lens 4

In each of the above drawings, the horizontal axis indicates a position (mm) on the surface of the body surface. The vertical axis indicates the depth (mm) from the body surface. Here, the “body surface” corresponds to the surface of the skin of the living body. In the simulation, physical properties of water close to the skin are used.

In this simulation result, an ultrasonic wave intensity distribution of 0 kW/m² to 1.8 kW/m² is obtained. In each of the simulation results, the reference numeral SO indicates a region where the ultrasonic wave intensity was 0 kW/m² to 0.18 kW/m². The reference numeral S1 indicates a region where the ultrasonic wave intensity was 0.18 kW/m² to 0.36 kW/m². The reference numeral S2 indicates a region where the ultrasonic wave intensity was 0.36 kW/m² to 0.54 kW/m². The reference numeral S3 indicates a region where the ultrasonic wave intensity was 0.54 kW/m² to 0.72 kW/m². The reference numeral S4 indicates a region where the ultrasonic wave intensity was 0.72 kW/m² to 0.90 kW/m². The reference numeral S5 indicates a region where the ultrasonic wave intensity was 0.90 kW/m² to 1.08 kW/m². The reference numeral S6 indicates a region where the ultrasonic wave intensity was 1.08 kW/m² to 1.26 kW/m². The reference numeral S7 indicates a region where the ultrasonic wave intensity was 1.26 kW/m² to 1.44 kW/m². The reference numeral S8 indicates a region where the ultrasonic wave intensity was 1.44 kW/m² to 1.62 kW/m². The reference numeral S9 indicates a region where the ultrasonic wave intensity was 1.62 kW/m² to 1.80 kW/m². In addition, in a part where a plurality of regions in which the ultrasonic wave intensity continuously increases are concentrated so as to form an elliptical shape, the plurality of regions may be collectively indicated as, for example, “S3 to S9”.

### <Test Device 80>

As shown in FIG. 17, the test device 80 includes a computer 81, a display 82, an oscilloscope 83, a movable stage 84, an ultrasonic wave generation unit 85, a hydrophone 86, a device 87, and a water tank 88.

The computer 81 includes a control unit that comprehensively controls the test device 80, and a storage unit that stores conditions and test results of the lenses 1 to 4. The display 82 displays the conditions and test results of the lenses 1 to 4. The display 82 is connected to the computer 81. The oscilloscope 83 is connected to the hydrophone 86 and acquires signal data detected by the hydrophone 86. The oscilloscope 83 is connected to the computer 81. The movable stage 84 can move the hydrophone 86 in a horizontal direction under the control of the computer 81. The ultrasonic wave generation unit 85 includes, for example, a function generation unit that generates an electrical signal such as a sine wave or a rectangular wave, and an amplifier. The ultrasonic wave generation unit 85 corresponds to, for example, the device body 100 shown in FIG. 1. The hydrophone 86 is, for example, a sensor that measures a sound field of the ultrasonic wave in water. The device 87 can be replaced and connected to the ultrasonic wave generation unit 85. The number of types of the device 87 is four in accordance with the lenses 1 to 4. Each of the four devices 87 includes one lens selected from the lenses 1 to 4 and a piezoelectric element. The device 87 corresponds to the ultrasonic wave unit. Water is contained in the water tank 88. In the water of the water tank 88, the device 87 and the hydrophone 86 are arranged to face each other.

In such a test device 80, while moving the hydrophone 86 in the horizontal direction by 0.5 mm by the driving of the movable stage 84, the ultrasonic wave intensity at the position after the movement was measured.

### <Experimental Results Using Test Device 80>

Each of FIGS. 19, 21, 23, 25, 27, and 29 shows the following experimental results using the test device 80.

FIG. 19: experimental results of Comparative Example in which only a piezoelectric element having a diameter of 6 mm was used without using a lens (FZP member)

FIG. 21: experimental results of the lens 1

FIG. 23: experimental results of the lens 2

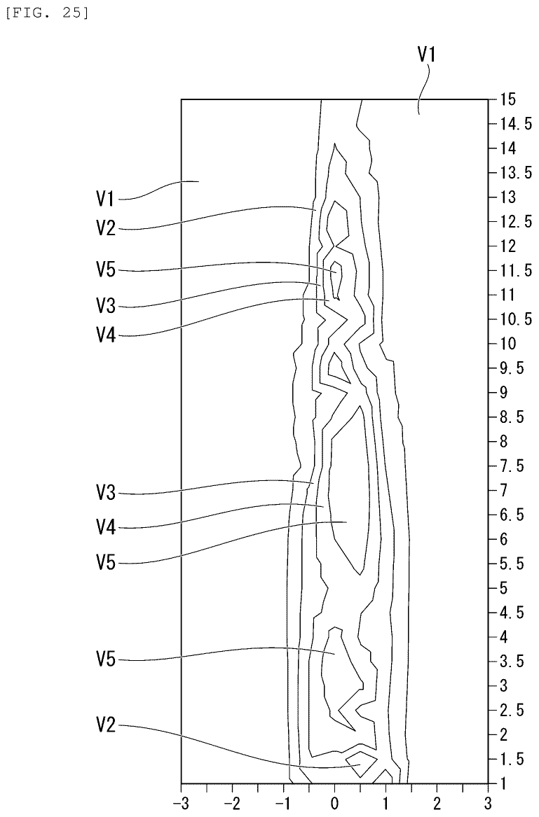

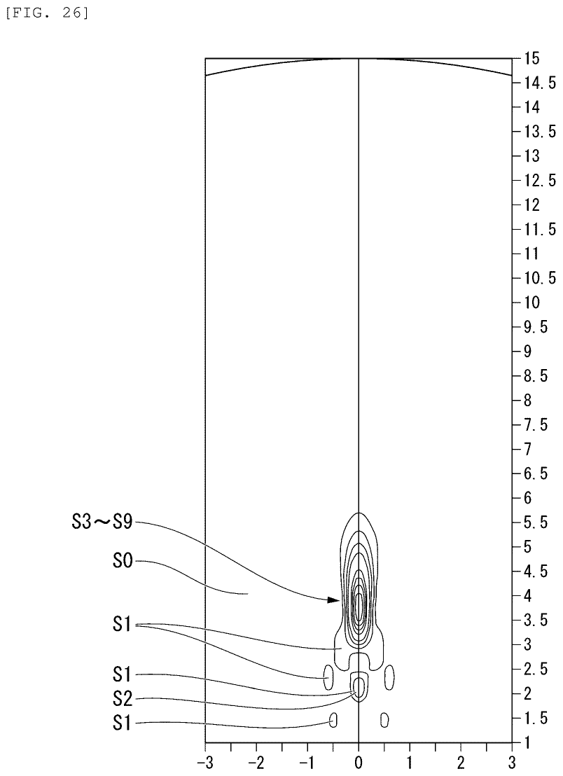

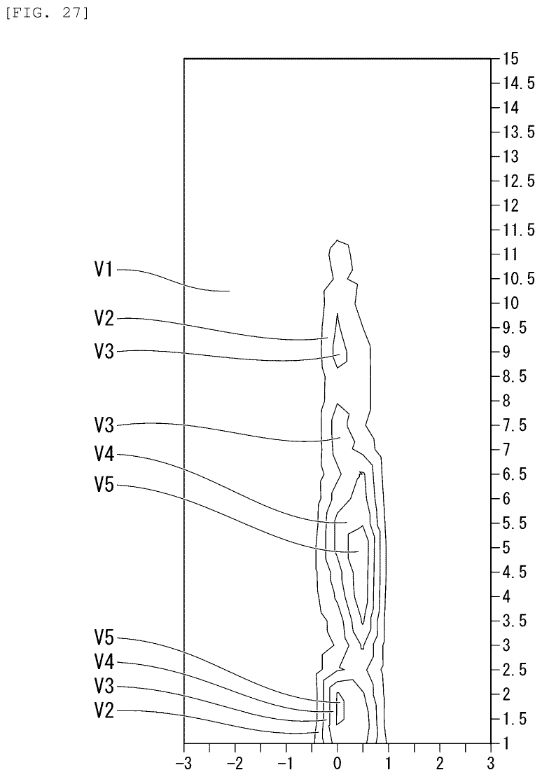

FIG. 25: experimental results of Comparative Example in which only a piezoelectric element having a diameter of 5 mm was used without using a lens (FZP member)

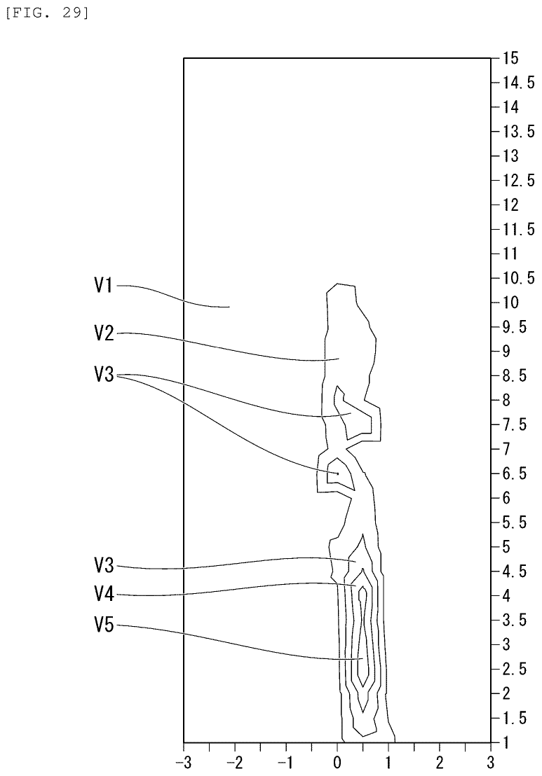

FIG. 27: experimental results of the lens 3

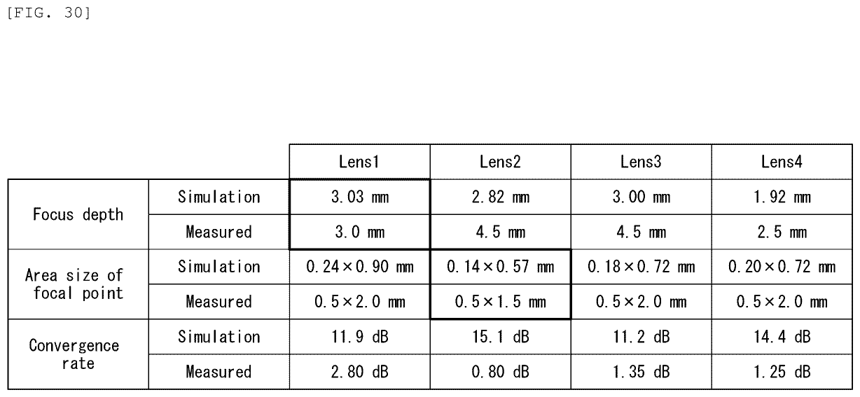

FIG. 29: experimental results of the lens 4

In each of the above drawings, the horizontal axis indicates a position (mm) on the surface of the body surface. The vertical axis indicates the depth (mm) from the body surface. Here, the “body surface” corresponds to the surface of the skin of the living body. Water is used in the test device 80. The reason for using water is that the physical properties of water is close to the skin. The “body surface” corresponds to an interface between the device 87 and water. In other words, it corresponds to the interface between the lens and water.

In this experimental result, each of the plurality of measurement values is normalized. That is, a maximum value is extracted from a plurality of measurement values indicating the ultrasonic wave intensity, and a plurality of calculation values are obtained by dividing each of the plurality of measurement values by the maximum value. Therefore, the normalized calculation values are in the range of 0 to 1.0. In each of FIGS. 19, 21, 23, 25, 27, and 29, the reference numeral V1 indicates a part in which the calculation values were 0 to 0.2. The reference numeral V2 indicates a part in which the calculation values were 0.2 to 0.4. The reference numeral V3 indicates a part in which the calculation values were 0.4 to 0.6. The reference numeral V4 indicates a part in which the calculation values were 0.6 to 0.8. The reference numeral V5 indicates a part in which the calculation values were 0.8 to 1.0.

### SUMMARY

FIG. 30 is a table summarizing the simulation results (Simulation) and experimental results (Measured) of the lenses 1 to 4 relating to Example 4.

FIG. 30 shows the focus depth, the area size of focal point, and the convergence rate. Here, the area size of focal point means a range irradiated with an intensity of 50% or more of the maximum intensity. The convergence rate (dB) is a value that is compared with the maximum intensity of each of the lenses 1 to 4 with respect to the maximum intensity of Comparative Example in which the FZP member is not mounted.

### (Evaluation Results)

From the results shown in FIGS. 18 to 30, the following points are clear.

(E1) In Comparative Example, the ultrasonic wave intensity is distributed over the entire depth from the body surface. Therefore, it is clear that the ultrasonic wave cannot be focused at a high intensity on the target depth.

(E2) In each of the lenses 1 to 4, it was possible to obtain a distribution in which the ultrasonic wave converges with a high intensity. Therefore, in each of the lenses 1 to 4, it was confirmed that a focal point was created as assumed.

(E3) In Example 4, the FZP member shown in FIG. 3B is used. The FZP member includes four coupling portions 23. It is clear that the experimental results close to the simulation results were obtained regardless of the presence or absence of the four coupling portions 23.

(E4) It is clear that, with respect to the focus depth of the lens 1, the simulation results and the experimental results were almost the same result. That is, a target value of 3.0 mm can be achieved.

(E5) Concerning the convergence rate of the lens 1, the experimental result was approximately 3.0 dB, and it is clear that a high convergence rate can be obtained and that the experimental result can be brought close to the simulation result.

(E6) The area size of focal point of the lens 2 can be reduced in both the simulation result and the experimental result.

(E7) Each of the lenses 1 and 2 has a diameter of 6.0 mm, and each of the lenses 3 and 4 has a diameter of 5.0 mm. That is, the lenses 3 and 4 are smaller than the lenses 1 and 2. It is clear that due to such a difference in size, it is difficult to adjust the focusing characteristics of the lenses 3 and 4.

### REFERENCE SIGNS LIST

1, 1A, 3, 4, 5, 6 ultrasonic wave unit
10 piezoelectric element
10F ultrasonic wave generation surface
20 FZP member (transmissive diffraction portion)
20F first FZP surface (first surface)
20S second FZP surface (second surface)
21 inner FZP portion
22 outer FZP portion
23 coupling portion
25 slit
30 gel member
30A first gel member
30B second gel member
30F contact surface
30S exposed surface
31 first urethane resin
31A upper surface
32 second urethane resin
32A liquid surface
35 first gel region
36 second gel region
40 application gel
50, 60 diffraction gel tape (diffraction swelling tape)
50F, 60F first tape surface
50S, 60S second tape surface
51F first protective sheet
51S second protective sheet
55 diffraction gel member
70 base
70F base surface
71 frame
71N frame inner surface
72 mold
80 test device
81 computer
82 display
83 oscilloscope
84 movable stage
85 ultrasonic wave generation unit
86 hydrophone
87 device
88 water tank
100 device body
101 AC voltage generation unit
102 signal generation unit
103 signal cable
200 ultrasonic wave focusing device
P1 center opening
P2 peripheral opening
SP slit pattern

1. An ultrasonic wave unit, comprising:
a piezoelectric element having an ultrasonic wave generation surface for generating an ultrasonic wave; and
a transmissive diffraction portion positioned on the ultrasonic wave generation surface or away from the ultrasonic wave generation surface.

2. The ultrasonic wave unit according to claim 1, wherein
the transmissive diffraction portion is positioned on the ultrasonic wave generation surface, and
the transmissive diffraction portion is a member different from the piezoelectric element.

3. The ultrasonic wave unit according to claim 1, further comprising:
a swelling body provided on the ultrasonic wave generation surface to cover the transmissive diffraction portion.

4. The ultrasonic wave unit according to claim 1, wherein
the transmissive diffraction portion is positioned away from the ultrasonic wave generation surface,
the transmissive diffraction portion has a first surface facing and away from the ultrasonic wave generation surface and a second surface opposite to the first surface,
a swelling body is disposed at least between the ultrasonic wave generation surface and the first surface, and
the swelling body is configured to adhere at least the transmissive diffraction portion to the piezoelectric element.

5. The ultrasonic wave unit according to claim 4, wherein
the swelling body is configured to adhere the transmissive diffraction portion to the piezoelectric element to cover both the first surface and the second surface.

6. The ultrasonic wave unit according to claim 4, wherein
the swelling body includes
a first swelling body positioned between the ultrasonic wave generation surface and the first surface and configured to adhere the transmissive diffraction portion to the piezoelectric element, and
a second swelling body attachable to and detachable from the second surface.

7. The ultrasonic wave unit according to claim 4, wherein
the swelling body has a contact surface that is in contact with the ultrasonic wave generation surface, and an exposed surface that is a surface opposite to the contact surface and that is exposed to an outside of the ultrasonic wave unit,
in a direction from the contact surface toward the exposed surface, the swelling body positioned between the first surface and the contact surface has a first thickness, and the swelling body positioned between the second surface and the exposed surface has a second thickness, and
by adjusting at least one of the first thickness and the second thickness, a focal length from the exposed surface to a focal point or a convergence rate of the ultrasonic wave is adjusted.

8. The ultrasonic wave unit according to claim 4, wherein
the swelling body has a contact surface that is in contact with the ultrasonic wave generation surface, and an exposed surface that is a surface opposite to the contact surface and that is exposed to an outside of the ultrasonic wave unit,
the transmissive diffraction portion has a slit, and
by adjusting a width of the slit, a focal length from the exposed surface to a focal point or a convergence rate of the ultrasonic wave is adjusted.

9. The ultrasonic wave unit according to claim 1, wherein
the transmissive diffraction portion includes
a first member,
a second member away from the first member and surrounding the first member, and
a coupling portion positioned between the first member and the second member and coupling the first member to the second member.

10. (canceled)

11. (canceled)

12. The ultrasonic wave unit according to claim 2, further comprising:
a swelling body provided on the ultrasonic wave generation surface to cover the transmissive diffraction portion.

13. The ultrasonic wave unit according to claim 5, wherein
the swelling body has a contact surface that is in contact with the ultrasonic wave generation surface, and an exposed surface that is a surface opposite to the contact surface and that is exposed to an outside of the ultrasonic wave unit,
in a direction from the contact surface toward the exposed surface, the swelling body positioned between the first surface and the contact surface has a first thickness, and the swelling body positioned between the second surface and the exposed surface has a second thickness, and
by adjusting at least one of the first thickness and the second thickness, a focal length from the exposed surface to a focal point or a convergence rate of the ultrasonic wave is adjusted.

14. The ultrasonic wave unit according to claim 6, wherein
the swelling body has a contact surface that is in contact with the ultrasonic wave generation surface, and an exposed surface that is a surface opposite to the contact surface and that is exposed to an outside of the ultrasonic wave unit,
in a direction from the contact surface toward the exposed surface, the swelling body positioned between the first surface and the contact surface has a first thickness, and the swelling body positioned between the second surface and the exposed surface has a second thickness, and
by adjusting at least one of the first thickness and the second thickness, a focal length from the exposed surface to a focal point or a convergence rate of the ultrasonic wave is adjusted.

15. The ultrasonic wave unit according to claim 5, wherein
the swelling body has a contact surface that is in contact with the ultrasonic wave generation surface, and an exposed surface that is a surface opposite to the contact surface and that is exposed to an outside of the ultrasonic wave unit,
the transmissive diffraction portion has a slit, and
by adjusting a width of the slit, a focal length from the exposed surface to a focal point or a convergence rate of the ultrasonic wave is adjusted.

16. The ultrasonic wave unit according to claim 6, wherein
the swelling body has a contact surface that is in contact with the ultrasonic wave generation surface, and an exposed surface that is a surface opposite to the contact surface and that is exposed to an outside of the ultrasonic wave unit,
the transmissive diffraction portion has a slit, and
by adjusting a width of the slit, a focal length from the exposed surface to a focal point or a convergence rate of the ultrasonic wave is adjusted.

17. A diffraction swelling tape to be used in the ultrasonic wave unit according to claim 1, the diffraction swelling tape comprising:
a transmissive diffraction portion having a first surface and a second surface opposite to the first surface; and
a swelling body configured to cover at least one of the first surface and the second surface and to adhere to a piezoelectric element.

18. An ultrasonic wave focusing device comprising:
an ultrasonic wave unit;
a signal generation unit configured to supply a frequency signal to the ultrasonic wave unit; and
an AC voltage generation unit configured to supply an AC voltage to the signal generation unit, wherein
the ultrasonic wave unit includes
a piezoelectric element having an ultrasonic wave generation surface for generating an ultrasonic wave, and
a transmissive diffraction portion positioned on the ultrasonic wave generation surface or away from the ultrasonic wave generation surface
